(19) United States
(12) Patent Application Publication
(10) Pub. No.: US 2023/0397945 A1
(43) Pub. Date: Dec. 14, 2023

(54) POWER APPARATUS, CONTROL AND INVERTERS FOR ELECTROSURGERY

(71) Applicant: THE BOARD OF TRUSTEES OF THE UNIVERSITY OF ILLINOIS, URBANA, IL (US)

(72) Inventors: Sudip Mazumder, Chicago, IL (US); Congbo Bao, Chicago, IL (US)

(73) Assignee: THE BOARD OF TRUSTEES OF THE UNIVERSITY OF ILLINOIS, URBANA, IL (US)

(21) Appl. No.: 18/209,979

(22) Filed: Jun. 14, 2023

Related U.S. Application Data
(60) Provisional application No. 63/443,277, filed on Feb. 3, 2023.
(60) Provisional application No. 63/352,046, filed on Jun. 14, 2022.

(51) Int. Cl. A61B 18/12; A61B 18/14; A61B 18/16

(52) U.S. Cl. CPC A61B 18/1233; A61B 18/1402; A61B 18/16; A61B 2018/126; A61B 2018/00702

(57) ABSTRACT

The disclosure provides an example electrosurgical system and methods for use thereof. The electrosurgical system includes a high-frequency inverter (“HFI”) having a full bridge and a control system electrically coupled to the HFI. The control system controls output parameters including one or more of an output power Pₒᵤₜ(t) and an output voltage or current by varying power reference P_{ref}(t) or switch states of the HFI. The control system causes a power adaptation ΔP(t) to a preset power set Pₛₑₜ based on receiving at least one of impedance feedback and thermal feedback according to the following relationship: P_{ref}(t)=Pₛₑₜ+ΔP(t).

### CROSS-REFERENCE TO RELATED APPLICATIONS

This application is a U.S. Non-Provisional application that claims priority to U.S. Provisional Patent Application No. 63/352,046, filed Jun. 14, 2022, and to U.S. Provisional Patent Application No. 63/443,277, filed Feb. 3, 2023, which are hereby incorporated by reference in their entirety.

### STATEMENT OF GOVERNMENTAL INTEREST

This disclosure was made with government support from the National Institute of Biomedical Imaging and Bioengineering of the National Institutes of Health (NIH) under award number R01EB029766. The government has certain rights in the disclosure.

### BACKGROUND

Morton, in 1881, showed that human being's muscle stimulation terminates whenever the applied signal frequency is higher than 100 kHz. Based on that, electrosurgery was first invented in 1926 by William Bovie. Modern electrosurgery passes alternating signals with a fundamental frequency above 200 kHz but lower than 5 MHz through the human body to conduct clinical treatment, such as cutting, coagulation, and fulguration, etc. Therefore, inverters with high frequency (“HF”) outputs are required for electrosurgery.

Resonant inverters (e.g., class E, F, EF2, Class Φ₂) and their variants feature HF output with reasonable efficiency. However, none of those topologies has been investigated in the area of electrosurgery due to certain limitations, such as load sensitivity, high device voltage stress, and sophisticated tuning processes. Wide-bandgap (WBG) (e.g., SiC, GaN) device-based pulse-width modulation (“PWM”) inverters can operate at HF. However, to generate a fundamental (inverter-sinusoidal-output) frequency of at least 200 kHz, as needed by electrosurgery, an extremely high switching frequency (multi-MHz) is needed that is impractical from efficiency, thermal, and electromagnetic interference standpoints. Selective harmonic elimination (“SHE”) was pursued in line-frequency-commutated converters decades back owing to very-slow thyristor and triacs.

However, introduction of notches is still required for extracting the fundamental-frequency output at an acceptable total harmonic distortion (“THD”) and hence application of SHE will still require an electrosurgery HF inverter to operate at frequencies that are multiples of 200 kHz yielding similar challenges mentioned above.

The existing inverters for electrosurgery include inverters that generate square-wave output with a much higher switching frequency that yields higher switching loss and potentially tissue-damaging super-harmonics. Alternative known inverters also include soft-switched inverters that reduce the switching loss but can only support very narrow load range (to achieve soft switching) and, the load range bounds are also well short of the ones needed to support electrosurgery.

### SUMMARY

In a first aspect, an example electrosurgical system is disclosed. The electrosurgical system includes (a) a high-frequency inverter (“HFI”) having a full bridge, and (b) a control system electrically coupled to the HFI that controls output parameters including one or more of an output power Pₒᵤₜ(t) and an output voltage or current by varying power reference P_{ref}(t) or switch states of the HFI. The control system causes a power adaptation ΔP(t) to a preset power Pₛₑₜ based on receiving at least one of impedance feedback and thermal feedback according to the following relationship: P_{ref}(t)=Pₛₑₜ+ΔP(t).

In a second aspect, an example method for using the electrosurgical system according to the first aspect of the disclosure is provided. The method includes (a) receiving, via the control system, at least one signal with an indication of thermal feedback and/or impedance feedback, (b) determining, via the control system, a power adaptation ΔP(t) based on the thermal feedback and/or impedance feedback, and (c) combining, via the control system, a preset power Pₛₑₜ for the HFI with the determined power adaptation ΔP(t) to obtain the output power reference P_{ref}(t) for the HFI.

In a third aspect, an example non-transitory computer-readable medium having stored thereon program instructions that upon execution by a processor, cause performance of a set of steps according to the second aspect of the disclosure is provided. The non-transitory computer-readable medium includes (a) the control system receiving at least one signal with an indication of thermal feedback and/or impedance feedback, (b) the control system determining a power adaptation ΔP(t) based on the thermal feedback and/or impedance feedback, (c) the control system combining a preset power Pₛₑₜ for the HFI with the determined power adaptation ΔP(t) to obtain the output power reference P_{ref}(t) for the HFI.

Exemplary high-frequency inverters adapted for use in electrosurgery and associated exemplary power systems are set forth. Systems herein facilitate control of output parameters, such as output power, voltage, current etc., by varying power switch states. The power switch states can be based on one or more of a proper combination of impedance, spatial, and/or temporal feedback, estimations, and/or predictions, in order to sufficiently regulate collateral tissue damage during electrosurgery.

Systems and methods constructed in accordance with the principles of the disclosure can be configured to monitor load properties, such as load temperature, load impedance, etc. in an ultrafast real-time manner. High-frequency inverters constructed in accordance with the principles of the disclosure can be safely packaged into a case with output terminals and a normal AC plug as input. These high-frequency inverters can also be operated by professional surgeons during electrosurgery according to their clinical needs.

Exemplary high-frequency inverters set forth in the disclosure eliminate the need for an extremely high switching frequency (multi-MHz) to generate a fundamental (inverter-sinusoidal-output) frequency of at least 200 kHz, as needed by electrosurgery. Furthermore, the new control system set forth herein significantly and autonomously reduces collateral damage of electrosurgery. Systems and inverter devices herein eliminate potential tissue-damaging super-harmonics from the known square-waveform by incorporating sinewave generating technology. Systems herein further reduce electro-magnetic interference (“EMI”) during surgery. Power control systems for inverters herein can be based on thermal feedback, impedance feedback, model prediction, and/or additional parameters to reduce collateral tissue damage during electrosurgery. Systems herein can incorporate technology and filter architecture that supports a nominal 390 kHz output frequency (with added frequency bandwidth for supporting other modes) and variable output voltages depending on the modes of electrosurgery.

Exemplary systems herein eliminate the need of selective harmonic elimination (“SHE”) and avoid solving transcendental equations without requiring complex, or very-high-frequency PWM or expensive solutions.

Systems herein can incorporate one or more bandpass filters that have very high attenuation for both higher order harmonics and low frequency, and thus the muscle stimulation and waveform distortion due to low frequency and high frequency are avoided at the same time.

Systems herein can include one or more filters that produce a small gain at both high-order harmonics (e.g., 3rd, 5th, and 7th, etc.) and frequencies lower than 100 kHz, which distinguishes an exemplary multi-resonant frequency (“MRF”) filter from conventional low pass filters, such as LCL, and LLCL, etc.

The features, functions, and advantages that have been discussed can be achieved independently in various examples or may be combined in yet other examples further details of which can be seen with reference to the following description and drawings.

### BRIEF DESCRIPTION OF THE DRAWINGS

FIG. 1 is a functional block diagram of an electrosurgical system, according to one example implementation;

FIG. 2 shows a flowchart of a method for using the electrosurgical system, according to an example implementation;

FIG. 3 depicts a high-frequency inverter (i.e., transformer) T and an electric scalpel ES. Human tissue (i.e., the load) is placed between the electric scalpel and the return pad to complete the current return path, according to an example implementation;

FIG. 4 depicts the topology of a proposed VHFI. The high order filter (i.e., the multi-resonant frequency (“MRF”) filter) will pass the fundamental frequency element while blocking others, according to an example implementation;

FIG. 5 depicts ideal gate signals for a full bridge and an ideal output waveform of the high order filter, according to an example implementation;

FIG. 6 depicts a schematic of the high order filter, according to an example implementation;

FIG. 7 depicts a Bode plot of the high order filter with different load values, according to an example implementation;

FIG. 8 depicts the experimental setup of the very-high-frequency AC inverter without galvanic isolation, according to an example implementation;

FIG. 9 depicts continuous output voltage and current with the electric scalpel in pure cutting mode when a 100Ω load is connected. The maximum output voltage is about 260 V, and the peak current is about 2.3 A, according to an example implementation;

FIG. 10 depicts an oscilloscope FFT analysis frequency spectrum for output voltage at 100Ω with 300 W, according to an example implementation;

FIG. 11 depicts of blend cutting mode output voltage waveform, according to an example implementation;

FIG. 12 depicts an output voltage waveform for a blend cutting period, according to an example implementation.

FIG. 13 depicts a coagulation mode output voltage waveform. The T represents the fundamental output period, and n. T means the number of blank cycles between two bursts, according to an example implementation;

FIG. 14 depicts an output voltage waveform for the coagulation mode, according to an example implementation;

FIG. 15 depicts a waveform of experimental efficiency and total harmonic distortion (“THD”) versus different loads when output power is approximately maintained at 300 W, according to an example implementation;

FIG. 16 depicts a schematic of monopolar surgery using an electrosurgery generator (“ESG”). The high-frequency (“HF”) step-up transformer for galvanic isolation is abbreviated as T and the electric scalpel is noted as ES. The biomedical tissues are placed between the ES and the return pad to close the path for current flow. The surgeon selects the modes according to his/her electrosurgery needs, according to an example implementation;

FIG. 17 depicts the topology of the full-bridge HFI and MRF filter. The HFI employs phase-shift control to generate a bipolar square waveform which is shaped into a sinusoidal (fundamental) waveform by the MRF filter. Transformer turn ratio is designated as n and its secondary side is directly connected to the load. The output voltage regulation is achieved via adjustment of full-bridge phase-shift angle α, which is derived from the angular information of the feedforward output signal α₀ and the controller output signal Δα, according to an example implementation;

FIG. 18A depicts tank impedance for the fundamental frequency (390 kHz) and the short path is shown as a straight line.

FIG. 18B depicts tank impedance for the 3rd harmonic frequency, according to an example implementation;

FIG. 18C depicts tank impedance for the 5th harmonic frequency, according to an example implementation;

FIG. 18D depicts tank impedance for the 7th harmonic frequency, according to an example implementation;

FIG. 19 depicts actual- and nominal-inductance based Bode plots of the MRF filter, when it is directly connected to a 100Ω (or 380Ω) load (which is equivalent to a 570Ω (or 2.2 kΩ) load as seen from the secondary side if a transformer with a 2.396 turn ratio is connected), according to an example implementation;

FIG. 20 depicts the frequency spectrum of the HFI bipolar square waveform and the corresponding MRF filter attenuation, according to an example implementation;

FIG. 21 depicts the experimental setup of the HFI, according to an example implementation;

FIG. 22 depicts output voltage versus phase shift angle for three different DC inputs, according to an example implementation;

FIG. 23A depicts output THD for the HFI at different output powers that are adjusted by varying phase shift α and Vᵢₙ is set as 100 V, according to an example implementation;

FIG. 23B depicts output THD for the HFI at different output load resistances ranging from 300Ω to 4200Ω. Vᵢₙ is set as 100 V (200 V) for load resistances lower (higher) than 1 kΩ, according to an example implementation;

FIG. 24 depicts Output voltage regulation errors (R.E.) and THD at the different load resistances versus tank-1 capacitor deviating from its nominal value, according to an example implementation;

FIG. 25A depicts output start-up transient response of the HFI for a load resistance of 570Ω with Vᵢₙ and V_{ref} set at 100 V and 300 V, respectively, according to an example implementation;

FIG. 25B depicts output start-up transient response of the HFI for a load resistance of 2.2 kΩ with Vᵢₙ and V_{ref} set at 200 V and 600 V, respectively, according to an example implementation;

FIGS. 26A-B depicts output-voltage dynamics for a step change in the load. The load comprises a 300Ω (2200Ω) load resistor in parallel with a switched 570Ω (770Ω) load resistor. By default, the load is 300Ω (2200Ω) and when the switch is turned on, the load changes to 196Ω (570Ω). The purple trace (V_{g}) is the gate signal of the switch triggering the load step change with 15 V. FIG. 26A shows step change in the load from 300Ω to 196Ω at T₁ and 196Ω to 300Ω at Ta, respectively, with Vᵢₙ as 100 V. FIG. 26B shows step change in the load from 2200Ω to 570Ω at T₁ and 570Ω to 2200Ω at T₂, respectively, with Vᵢₙ as 200 V. Output current for a load of 2200Ω is modestly distorted due to loading effect of the switch parasitics and the normal steady-state current is shown in FIGS. 26A-B, according to an example implementation;

FIG. 27 depicts a schematic of a thermal-feedback-based monopolar electrosurgery using an ESG. The current- and voltage-sensing transformers are denoted as CT and PT, respectively. The HF transformer (T) boosts the output voltage (Vₒᵤₜ(t)). The tissue load is placed between the electric scalpel (ES) and return pad. The thermal camera monitors tissue surface temperature, according to an example implementation;

FIG. 28 depicts a schematic of the impact on a tissue load of an ill-suited power setting, according to an example implementation;

FIG. 29 depicts output power control system for the HFI, according to an example implementation. The power controller tracks the power reference (P_{ref}(t)). The thermal-feedback-based power adaption control autonomously modifies the preset power (Pₚᵣₑ₋ₛₑₜ) via a power adaption (ΔP(t)). ADC is the analog to digital converter module. Digital signal processor (DSP) used here is TMS320F28379D. Thermal sensor is 24×32 pixels. Tₜᵢₛₛᵤₑ(t) is an array of dimension 24×32 that represents the spatio-temporally sensed tissue surface temperature while Tₜᵢₛₛᵤₑ^{max} (t) is the maximum of Tₜᵢₛₛᵤₑ(t) in each sensing refresh frame. Data transmission delay (τ) is negligible;

FIG. 30 depicts an experimental setup of the HFI. Programmable Emile3 3-axes robotic gantry is employed to hold the ES and move it in a specified direction and at a uniform speed for the repeatability of the biomedical tissue cutting experiments, according to an example implementation;

FIG. 31 depicts the statistical distribution of output power (P̅ₒ(t)). Symbol (P̅ₒ(t)) represents the overall average of P̅ₒ(t) but spread over all of the 200 samples, according to an example implementation;

FIG. 32A depicts a top view of a tissue load with a plurality of cuts showing cut gaps and thermal spread, according to an example implementation;

FIG. 32B depicts a front view of the tissue load of FIG. 32A, according to an example implementation;

FIG. 33A depicts adaptive power reference that comes into effect at tₒ, where the maximum tissue surface temperature Tₜᵢₛₛᵤₑ(t) exceeds 40° C. (slightly higher than normal body temperature) and ends at t₁, according to an example implementation;

FIG. 33B depicts the maximum of sensed tissue surface temperature Tₜᵢₛₛᵤₑ(t) The cutting period starts at tₛₜₐᵣₜ and ends at t_{end}. The temperatures of test scenarios 1 and 2 fluctuate with a larger ripple due to lack of power adaptation. However, test scenarios with power adaptation, namely test scenarios 3-5, feature smaller temperature ripples and thus contribute to improved cutting uniformity. The hottest part of the ES is buried inside the tissue during the cutting process and the thermal sensor, under nominal condition, cannot observe it until the end of cutting. The ES tip is directly exposed to the thermal sensor after the cutting is completed, and thus a temperature spike occurs in the plot, which gradually decays afterwards, according to an example implementation;

FIG. 34A depicts low-frequency current paths inside a tissue load, according to an example implementation;

FIG. 34B depicts high-frequency current paths inside a tissue load, according to an example implementation;

FIGS. 35A-B depict the impact of thermal sensor mounting locations on temperature measurement. The thermal sensor used here is MLX90640 and its field of view during cutting is approximately illustrated as the teal shadow. FIG. 35A shows the sensor is mounted externally to the electrode. FIG. 35B shows the sensor is mounted together with the electrode.

FIG. 35C plots the obtained maximum tissue surface temperature for the thermal sensor mounting locations in FIGS. 35A-B. tₛₜₐᵣₜ and t_{end} indicate the beginning and end of cutting, respectively, according to an example implementation;

FIGS. 36A-C depict the linear load characteristics of the biotissue, according to an example implementation. FIG. 36A shows output current is in phase with voltage for pure resistive load. FIG. 36B shows current is leading θ° for capacitive load. FIG. 36C shows current is lagging θ° for inductive load;

FIG. 37A depicts a graphical representation of arcing during electrosurgery, according to an example implementation;

FIG. 37B depicts an experimental demonstration of arcing. The gauged dimension of the blade electrode used in Example 4 is marked on the righthand side, according to an example implementation;

FIG. 38A depicts a graphical representation of output voltage and distorted current based on the multi-sampling-based average power calculation. Output signals are sampled N times per output cycle Tₒ. The ADC sampling period is denoted as Tₛ, according to an example implementation;

FIG. 38B depicts a graphical representation of instant power obtained by multiplying voltage and current. With 28 sampling points per cycle, the obtained mean value of power is 29.9 W for sampled discrete signals versus 30.2 W for original continuous signals, according to an example implementation;

FIG. 39 depicts an illustration of electrocuting a tissue load from point A to B with a triangular cross-section. The central silver-gray layer represents tissue being removed, the middle red region designates tissues denaturized, while the outer pink zone witnesses the overheated area. The electrode insertion depth is denoted as h (m), while cutting width and cutting speed are specified as r (m) and v (m/s), respectively. The electrocuting duration zit illustrated here is extremely short, according to an example implementation;

FIG. 40 depicts a closed-loop control block diagram. The modulator outputs pulse-width modulation (“PWM”) signals that switch GaN devices in electrosurgery inverter (i.e., the HFI), according to an example implementation;

FIG. 41 depicts a graphical representations of an examination of the accuracy of the sparse-sampling-based power calculation at various wattages, namely (a) 15 W, (b) 25 W, (c) 35 W, (d) 45 W. The power value in the digital signal processor is denoted as DSP, while those produced by data from the digital storage oscilloscope are marked as DSO. The DSP used here is TMS320F28379D. The mean value over all 1000 samples is designated, according to an example implementation;

FIG. 42A depicts a graphical representation of output current that is barely distorted during electrocuting relative to output voltage that is shown with the higher magnitude, according to an example implementation;

FIG. 42B depicts a graphical representation of output current that is slightly distorted during electrocuting relative to output voltage that is shown with the higher magnitude, according to an example implementation;

FIG. 42C depicts a graphical representation of output current that is appreciably distorted during electrocuting relative to output voltage that is shown with the higher magnitude, according to an example implementation;

FIG. 42D depicts a graphical representation of output current versus voltage together with their total harmonic distortion, according to an example implementation;

FIG. 43 depicts a power accuracy test of the multi-sampling-based method. The values from DSP or DSO are separated by legend for various wattages, namely (a) 15 W. (b) 25 W. (c) 35 W. (d) 45 W, according to an example implementation;

FIG. 44A depicts a graphical representation of steady-state power tracking performance at 35 W. The output power is the trace in with the lowest magnitude, output current has the middle magnitude, and output voltage has the highest magnitude, according to an example implementation;

FIG. 44B depicts a graphical representation of a steady-state power tracking performance at 50 W, according to an example implementation;

FIG. 44C depicts a graphical representation of power steps from 35 W to 50 W, according to an example implementation;

FIG. 45A depicts a graphical representation of the secured load impedance defined in relationship (9) of Example 4 versus the electrode insertion depth. And then, parametric value of λ in (12) is around 54,000 while parameters α and β are equal to 10,700 and 9,100, respectively. However, the value of λ is reduced to 0.054 if the h·v is in the unit of m²/s, according to an example implementation;

FIG. 45B depicts a graphical representation of the midpoint of load impedance over 2000 samples versus electrode insertion depth that varies from 4 mm to 16 mm, according to an example implementation;

FIG. 45C depicts a graphical representation of the load impedance versus the electrode moving speed v, according to an example implementation;

FIG. 46A depicts a photograph of a top view of a tissue load with five electrocuting traces, according to an example implementation;

FIG. 46B depicts a photograph of a front view of the tissue load in FIG. 46A, according to an example implementation;

FIG. 46C depicts a photograph of a left side view of a tissue load in FIG. 46A, according to an example implementation;

FIG. 47A depicts graphical representations of a fragment of load impedance and power reference P_{ref}(t) and average output power P̅ₒ(t) per cycle captured from trace 2 in the tissue load in FIGS. 46A-C, according to an example implementation;

FIG. 47B depicts graphical representations of a fragment of load impedance and power reference P_{ref}(t) and average output power P̅ₒ(t) per cycle captured from trace 3 in the tissue load in FIGS. 46A-C, according to an example implementation;

FIG. 47C depicts graphical representations of a fragment of load impedance and power reference P_{ref}(t) and average output power P̅ₒ(t) per cycle captured from trace 4 in the tissue load in FIGS. 46A-C, according to an example implementation; and

FIG. 47D depicts graphical representations of a fragment of load impedance and power reference P_{ref}(t) and average output power P̅ₒ(t) per cycle captured from trace 5 in the tissue load in FIGS. 46A-C, according to an example implementation.

The drawings are for the purpose of illustrating examples, but it is understood that the disclosure is not limited to the arrangements and instrumentalities shown in the drawings.

### DETAILED DESCRIPTION

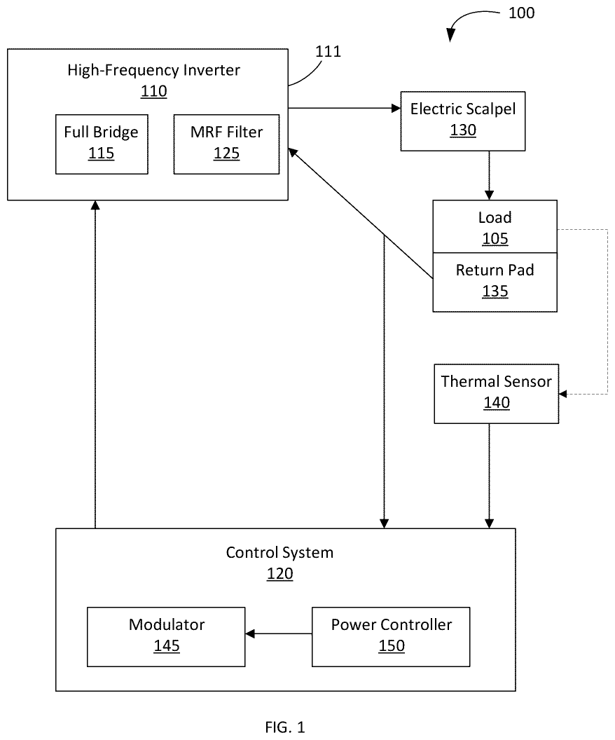

In a first aspect of the disclosure, shown in FIGS. 1, 3-4, 6, 16-18D, 27-29, 40, an electrosurgical system 100 for operating on a tissue load 105 includes a high-frequency inverter (“HFI”) 110 having a full bridge 115. The electrosurgical system 100 also includes a control system 120 electrically coupled to the HFI 110 that controls output parameters including one or more of an output power Pₒᵤₜ(t) and an output voltage or current by varying power reference P_{ref}(t) or switch states of the HFI 110. As used herein, “electrically coupled” refers to coupling using a conductor, such as a wire or a conductible trace, as well as inductive, magnetic, and wireless couplings. The control system 120 causes a power adaptation ΔP(t) to a preset power Pₛₑₜ based on receiving at least one of impedance feedback and thermal feedback according to the following relationship: P_{ref}(t)=Pₛₑₜ+ΔP(t).

In one optional implementation, the electrosurgical system 100 further includes a multi-resonant-frequency (“MRF”) filter 125 electrically coupled to the HFI 110. The MRF filter 125 includes a first resonant tank 126 and a second resonant tank 127. The first resonant tank 126 resonates at a switching frequency and the second resonant tank 127 resonates at least at third-, fifth-, and seventh-order harmonic. A fundamental output frequency of the HFI 110 is the same as a switching frequency of the HFI 110. In one optional implementation, the switching frequency is 390 kHz. The MRF filter 125 is discussed in more detail in Example 1 and 2 below.

In one optional implementation, the HFI 110 generates a bipolar square waveform, and the MRF filter 125 shapes the bipolar square waveform into a sinusoidal waveform output. Further, a transformer primary side voltage of the HFI 110 is determined based on the following:

$${V}_{p} ( t ) = \frac{4 {V}_{i n}}{π} \cdot cos ( α ) \cdot sin ( 2 π {f}_{s} t ) .$$

In one optional implementation, the electrosurgical system 100 also includes an electric scalpel 130 electrically coupled to a transformer secondary side 111 of the HFI 110 and a return pad 135 electrically coupled to the transformer secondary side 111 of the HFI 110. The return pad 135 is configured to receive a load 105 in the form of biomedical tissue that permits current flow therethrough from the electric scalpel 130 to the return pad 135 thereby closing a path for the current flow.

In one optional implementation, the electrosurgical system 100 additionally includes a thermal sensor 140 electrically coupled to the control system 120. The thermal sensor 140 is configured to detect a surface temperature of the load 105. The thermal sensor 140 may include an infrared sensor. The thermal sensor 140 is discussed in more detail in Example 3 below.

In one optional implementation, the control system 120 further includes a modulator 145 configured to output pulse-width modulation signals to the HFI 110, and a power controller 150 that tracks the output power reference P_{ref}(t). The power controller is discussed in more detail in Examples 3 and 4 below.

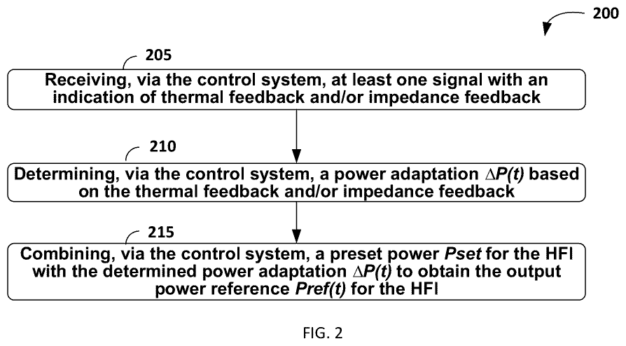

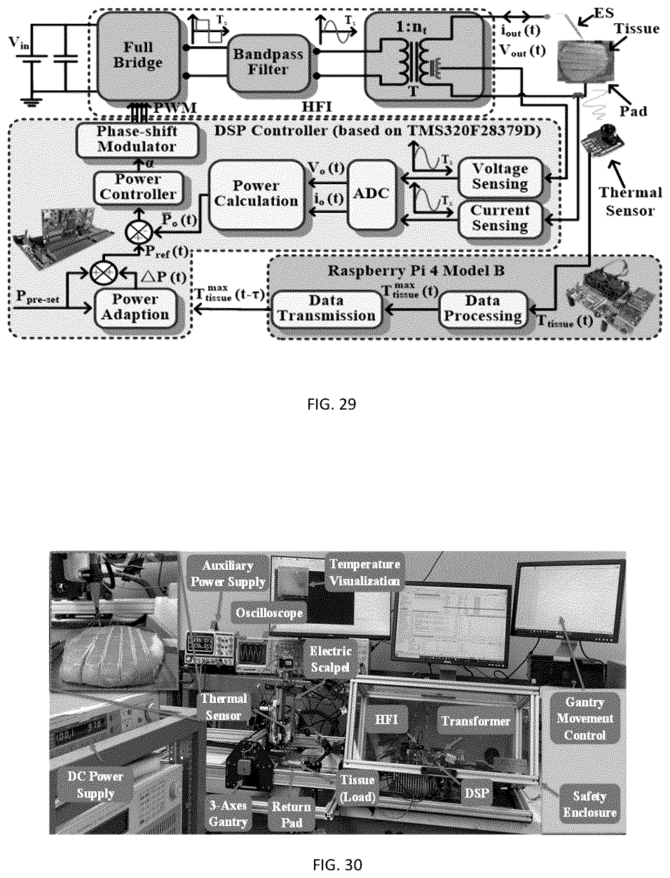

The control system further includes one or more processors that are detailed in Examples 3 and 4 below and as shown in FIGS. 29 and 40, for example. The processor(s) may be used to perform functions of the method shown in FIG. 2 and described below. The processor(s) may include a communication interface, data storage, an output interface, and a display each connected to a communication bus. The processor(s) may also include hardware to enable communication within each processor and between the processor(s) and other devices. The hardware may include transmitters, receivers, and antennas, for example.

The data storage may include or take the form of one or more computer-readable storage media that can be read or accessed by the processor(s). The computer-readable storage media can include volatile and/or non-volatile storage components, such as optical, magnetic, organic or other memory or disc storage, which can be integrated in whole or in part with the processor(s). The data storage is considered non-transitory computer readable media. In some examples, the data storage can be implemented using a single physical device (e.g., one optical, magnetic, organic or other memory or disc storage unit), while in other examples, the data storage can be implemented using two or more physical devices.

The data storage thus is a non-transitory computer readable storage medium, and executable instructions are stored thereon. The instructions include computer executable code. When the instructions are executed by the processor(s), the processor(s) are caused to perform functions.

The processor(s) may be a general-purpose processor or a special purpose processor (e.g., digital signal processors, application specific integrated circuits, etc.). The processor(s) may receive inputs from the communication interface and process the inputs to generate outputs that are stored in the data storage and output to the display. The processor(s) can be configured to execute the executable instructions (e.g., computer-readable program instructions) that are stored in the data storage and are executable to provide the functionality of the computing device described herein.

The following method 200 may include one or more operations, functions, or actions as illustrated by one or more of blocks 205-215. Although the blocks are illustrated in a sequential order, these blocks may also be performed in parallel, and/or in a different order than those described herein. Also, the various blocks may be combined into fewer blocks, divided into additional blocks, and/or removed based upon the desired implementation. Alternative implementations are included within the scope of the examples of the present disclosure in which functions may be executed out of order from that shown or discussed, including substantially concurrent or in reverse order, depending on the functionality involved, as would be understood by those reasonably skilled in the art.

Referring now to FIG. 2, a method 200 is illustrated for using the electrosurgical system 100 according to the first aspect of the disclosure. Method 200 includes, at block 205, receiving, via the control system 120, at least one signal with an indication of thermal feedback and/or impedance feedback. Then, at block 210, the control system 120 determines a power adaptation ΔP(t) based on the thermal feedback and/or impedance feedback. Next, at block 215, the control system 120 combines a preset power Pₛₑₜ for the HFI 110 with the determined power adaptation ΔP(t) to obtain the output power reference P_{ref}(t) for the HFI 110.

In one optional implementation, the control system 120 receiving the at least one signal with the indication of the thermal feedback and/or the impedance feedback includes the control system 120 receiving each switching cycle at least one signal indicating values for a plurality of pairs of output voltage and output current that are measured simultaneously during a given switching cycle. This process is discussed in more detail in Example 4 below with respect to multi-sampling-based power calculations.

In one optional implementation, the control system 120 monitors the output power Pₒᵤₜ(t) and thereby tracks the output power reference P_{ref}(t).

In one optional implementation, method 200 further includes the control system 120 determining an ideal average output power P_{idl} based on a cutting time duration Δt, a mass m of the load, a temperature rise ΔT of the load, a specific heat capacity c_{eq} of the load, a density ρ of the load, an electrode insertion depth h, and/or a cutting width r, as set forth below:

P_{idl}·Δt=m·c_{eq}·ΔT=½·r·h·v·Δt·ρ·c_{eq}·ΔT.

In one optional implementation, the control system 120 determining the power adaptation ΔP(t) based on the thermal feedback and/or impedance feedback includes the control system 120 determining a load impedance based on a largest value of sampled output voltage and output current for the given switching cycle. Then the control system 120 determines the power adaptation ΔP(t) based on the load impedance and the ideal average output power P_{idl}. In a further optional implementation, determining the power adaptation ΔP(t) is further based on a load impedance value determined from a moving average of the determined load impedance values over at least 10 switching cycles.

In one optional implementation, method 200 further includes the control system 120 updating the output power reference P_{ref}(t) for the HFI for each switching cycle in 3 μs or less.

In one optional implementation, the control system 120 receiving the at least one signal with the indication of the thermal feedback and/or the impedance feedback includes the control system 120 receiving, per each switching cycle, at least one signal indicating an output voltage Vₒ(t) corresponding to an output voltage positive peak at Tₛ/4 and a first and a second sample of output current. The first sample of output current iₒ(k) is measured between 0 and Tₛ/4 and the second sample of output current iₒ(k+1) is measured after the first sample output current such that the first and the second output current samples do not overlap in time. This process is discussed in more detail in Example 4 with respect to sparse-sampling-based power calculations.

In one optional implementation, method 200 further includes the HFI 110 generating a bipolar square waveform. The MRF filter 125 electrically coupled to the HFI 110 then shapes the bipolar square waveform into a sinusoidal waveform output. The MRF filter 125 includes a first resonant tank 126 and a second resonant tank 127. The first resonant tank 126 resonates at a switching frequency and the second resonant tank 127 resonates at least at third-, fifth-, and seventh-order harmonics. A fundamental output frequency of the HFI 110 is the same as a switching frequency of the HFI 110. In a further optional implementation, the method 200 further includes the control system 120 determining a transformer primary side voltage of the HFI 110 electrically coupled to the MRF filter 125 based on the following:

$${V}_{p} ( t ) = \frac{4 {V}_{i n}}{π} \cdot cos ( α ) \cdot sin ( 2 π {f}_{s} t ) .$$

This process is discussed in more detail below in Examples 1 and 2.

In one optional implementation, method 200 further includes the control system 120 adjusting a phase shift angle α₀ between gate signals of diagonal switch pairs Q₁-Q₄ of the HFI 110 based on the following relationship:

$${α}_{0} = f ( {V}_{ref} ) = \frac{180}{π} \cdot {cos}^{- 1} ( \frac{π \cdot {V}_{ref}}{4 \cdot n \cdot {V}_{i n}} ) .$$

In one optional implementation, method 200 further includes the control system 120 continuously monitoring a surface temperature of the load 105. The control system 120 then determines that the surface temperature of the load 105 differs from a predetermined nominal tissue temperature. Next, the control system adjusts the power reference P_{ref}(t) based on the relationships:

$${P}_{ref} ( t ) = {P}_{s e t} + Δ P ( t )$$

$$Δ P ( t ) = {P}_{s e t} \cdot ( \frac{{T}_{n o m}}{max ( {T}_{t i s s u e} ( t ) )} - 1 )$$

such that the surface temperature of the load is controlled towards the predetermined nominal tissue temperature. This process is discussed in more detail in Example 3 below.

In a third aspect of the disclosure, a non-transitory computer-readable medium having stored thereon program instructions that upon execution by an electrosurgical system 100 according to the first aspect of the disclosure and that has one or more processors may be utilized to cause performance of any functions of the foregoing methods according to the second aspect of the disclosure.

As one example, a non-transitory computer-readable medium having stored thereon program instructions that upon execution by a processor, cause performance of a set of steps includes a control system 120 receiving at least one signal with an indication of thermal feedback and/or impedance feedback. The control system 120 then determines a power adaptation ΔP(t) based on the thermal feedback and/or impedance feedback. Next, the control system 120 combines a preset power Pₛₑₜ for the HFI 110 with the determined power adaptation ΔP(t) to obtain the output power reference P_{ref}(t) for the HFI 110.

The following four example sections discuss experimental results and further inform aspects of the various components of the electrosurgical system 100.

### Example 1: GaN-HEMT Based Very-High-Frequency AC Power Supply for Electrosurgery

To power electrosurgery, a very-high-frequency AC inverter (“VHFI”) is required. In this example, a full-bridge based VHFI is proposed to enable electrosurgery. Its high-frequency output generating mechanism and the high order filter design are explained. To check the feasibility of the proposed VHFI, a 300 W Gallium Nitride High Electron Mobility Transistors (“GaN HEMT”) based experimental setup with 390 kHz output frequency, has been designed and implemented. Experimental efficiency and total harmonic distortion (“THD”) results are graphed for pure cutting mode. It turns out that maximum THD is less than 2.5% for the proposed VHFI. Further, recurring and burst experiment results are provided for blend cutting mode and coagulation mode, respectively. The experiment results show that the proposed VHFI has extreme fast-responding time for both blend cutting and coagulation mode, and crest factor is about 21 for coagulation mode. All experiment results together validate the feasibility of the proposed VHFI and also verify its capability of supporting different load values under different clinical modes.

### I. Introduction

GaN HEMT can dramatically improve switching speed, reduce switching loss and thus, GaN HEMT has found wide use in high-frequency applications since emerging in about 2004. Electrosurgery employs high-frequency current, ranging from hundreds kHz to several MHz, passed through human tissues to generate desired clinical effect, such as pure cutting, blend cutting and coagulation. It is obvious that power devices, supporting high frequency with low switching loss, are essential to electrosurgery. Consequently, electrosurgery generator (ESG) which powers electrosurgery, becomes one of target applications for GaN HEMT.

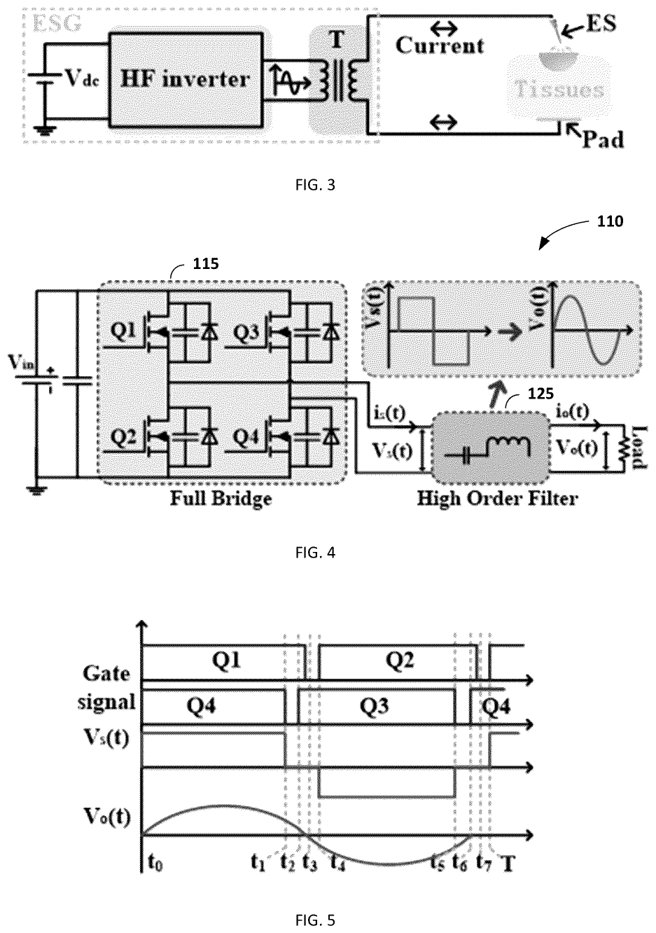

The illustration of electrosurgery using ESG has been highlighted in FIG. 3. During the electrosurgery, ESG acts as a high-frequency AC power supply and delivers preset energy to human tissues through electric scalpel, such that prospective surgical effect is produced. The energy delivered to human tissues will be manually set by surgeons before surgery and should be continuously adjusted according to actual human tissue type and expected clinical effect.

There are a large number of inverter topologies and a portion of them are grid-interfaced. The output frequency of these topologies is 50/60 Hz which is significantly slower than the device switching frequency and thus, many modulation schemes, such as sinusoidal pulse width modulation (SPWM), space vector pulse width modulation (SVPWM) can be applied. However, when it comes to high output frequency inverter, the fundamental output frequency is hundreds of kHz or several MHz, then SPWM and SVPWM modulation schemes will not be applicable anymore due to constraints from switching loss and thermal dissipation stress, etc. Therefore, soft-switching technique or different modulation schemes are highly necessary for high output frequency inverter. Class E inverter is well-known soft-switching based topology in high output frequency inverter family, and its output frequency can be more than 10 MHz while maintaining high efficiency. But it is worth noting that Class E inverter is load sensitive and voltage stress on power devices is quite high. A load-independent Class E inverter has been experimentally verified; however, the low and fixed output voltage makes it unsuitable for applications requiring variable high voltage. A Φ₂ inverter with lower device voltage stress has been described by others whereas the voltage stress across the device drain and source is still about 2.4 times of the input voltage. Yet, output THD is not mentioned by others, and also, output load is fixed.

ESG involves high frequency AC output with variable output voltage and varied load. Different output voltage magnitudes are required by different clinical purposes while the load variability during normal electrosurgery operation originates from two aspects. The first one is human tissues variation and the second one is plasma arcing dynamic transients. Different types of human tissues, such as skin, muscle and fat, etc. will characterize different electrical properties and tissue impedances will also change a lot even for the same piece of tissue as moving speed or moving direction of electric scalpel differs. The plasma arcing is in series with human tissues and will present when output voltage is sufficiently high such that the electric field around the electric scalpel exceeds that of the air. Further, plasma arcing is polarity dependent, therefore, Class E family, Φ₂ inverters and their variants are not attractive for electrosurgery case. Others have proposed a multiphase buck-based inverter with the capability of handling plasma arcing and modifying the output frequency. Yet, the output possesses square shape that contains significant harmonic components, and heavy electro-magnetic interference will be problematic. With the above-mentioned requirements in mind, this example depicts a GaN-HEMT based 390 kHz VHFI prototype capable of delivering energy to variable loads with adjustable output magnitude. At the same time, the output is of sinusoidal waveform, leading to low EMI emission, which makes the proposed prototype a good fit for electrosurgery application.

The rest part of this example is divided into four parts and organized as follows: In Section II, high-frequency AC output generation mechanism and the high order filter design are explained in detail. In Section III, GaN-based experiment validation for pure cutting mode, blend cutting mode, and coagulation mode are exhibited. Finally, this example is concluded in Section IV.

### II. High-Frequency Output Generation Mechanism and High Order Filter Design

A. High-Frequency AC Generation Mechanism

The topology capture of proposed VHFI is shown in FIG. 4 and it includes a full bridge block, a high order filter block, and a load. Even though the galvanic isolation part is of paramount importance and can isolate human tissues from unexpected grounding and prevent erroneous current path, it is not covered in this example.

Assuming that DC power supply, noted as Vᵢₙ, can support enough ripple power and the DC-link voltage is constant. Phase shift control strategy is employed here to switch the full bridge network at 390 kHz which is exactly the same as fundamental output frequency. For the initial understanding, mathematical analysis included here neglects all nonideal parasitics, such as DC-link voltage ripple, device conduction resistance, gate signal rising and falling time. The ideal gate signals, triggering GaN devices, are shown in FIG. 5. It is well-known that full bridge output equals to Vᵢₙ (−Vᵢₙ) when diagonal devices Q1(Q2) and Q4(Q3) are on at the same time, whereas the output will be zero if Q1 and Q3 or Q2 and Q4 are turned on simultaneously. The ideal output waveform of full bridge, Vₛ(t), is delineated as square waveform in FIG. 5 and can be expressed as superposition of trigonometric series as follows.

$${V}_{s} ( t ) = \frac{4 {V}_{i n}}{π} \cdot \frac{1}{n} {\sum }_{n = 1 , 3 , 5 , …}^{∞} sin ( 2 π {nf}_{s} t ) \qquad ( 1 )$$

Where n is the order of harmonics and only odd order frequency components exist for square wave. Vᵢₙ and ƒₛ are input voltage and switching frequency, respectively. In order to extract fundamental frequency of

$${V}_{s} ( t ) , \frac{4 {V}_{i n}}{π} sin ( 2 π {f}_{s} t ) ,$$

all higher order harmonics should be eliminated from Vₛ(t) and thus, the full bridge block is followed by a high order filter which should be able to pass 390 kHz whereas other harmonics are suppressed.

B. High Order Filter Design

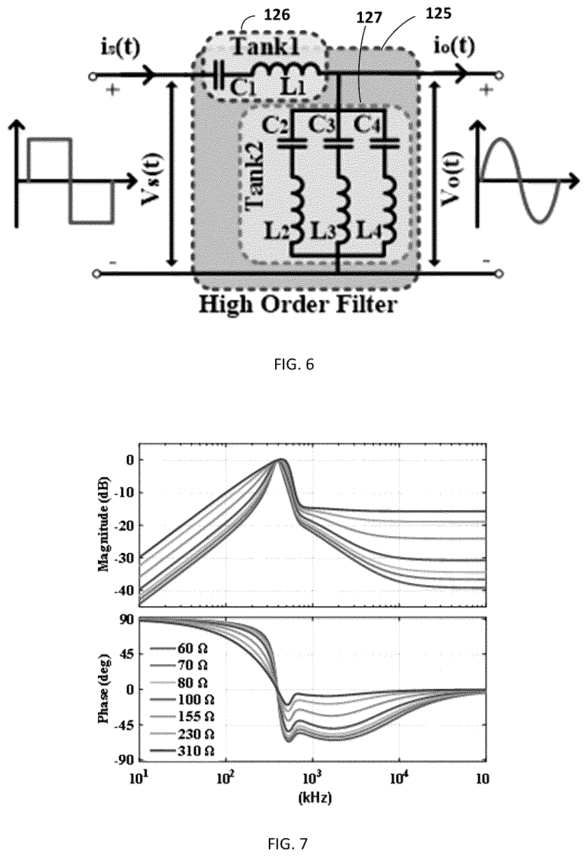

To extract the fundamental frequency from square-wave input, this paper proposes a high order filter structure, and its topology diagram is shown in FIG. 6. The high order filter is composed of two resonant tanks, marked as tank 1 and tank 2. Tank 1 is made of one inductor and one capacitor where their resonant center is tuned as the same with output that is 390 kHz in this paper. Tank 2 is made of three resonant legs and their resonant centers are placed at 3^{rd}, 5^{th}, and 7^{th} order harmonic frequency, separately. Hence, the fundamental frequency component will pass through tank 1 and compose of filter output. Regarding other high order harmonics, tank 1 and tank 2 will behave like a voltage divider, where major magnitude of high order harmonics will be held by tank 1 and only the minor part reaches tank 2. In consequence, high order harmonics are attenuated while fundamental frequency is maintained. In other words, the square wave input, Vₛ(t), will be shaped into sinusoidal output, Vₒ(t), of 390 kHz frequency.

As is known, filter bandwidth will enlarge and frequency selectivity will worsen as its quality factor reduces. As a result, more harmonic components will pass, and waveform distortion will deteriorate. To achieve better THD, high quality factor is preferred, however, high quality will also lead to high capacitor voltage stress, increase cost, and augment tuning difficulty. Therefore, the filter quality factor value of tank 1 and tank 2 should be determined and optimized by taking all aforementioned factors into consideration. This example targets loads ranging from 60Ω to 310Ω with maximum output power as 300 W and the quality factor is chosen as 0.82 for 300Ω, which should be further optimized between THD and inductor loss in the future. Given such high frequency, ferrite core inductors, featuring very high self-resonant frequency (“SRF”), are employed. Meanwhile, C0G dielectric capacitors with high voltage rating, extremely low equivalent resistance (“ESR”) and inductance (“ESL”) are of necessity here.

The transfer function of the proposed high order filter is derived and written in equation (2).

$$T ( s ) = \frac{{V}_{o} ( t )}{{V}_{s} ( t )} = \frac{{Z}_{tank 2} ( s ) \vert \vert R}{\{ {Z}_{tank 2} ( s ) \vert \vert R ) \} + {Z}_{tank 1} ( s )} \qquad ( 2 )$$

Where Zₜₐₙₖ₁(s) and Zₜₐₙₖ₂(S) is the impedance of tank 1 and tank 2, respectively. The load connected to the filter is represented as R and only pure resistive load is considered in this paper. Human tissue is not purely resistive and, therefore, more accurate load modeling is needed in future research.

To investigate the efficacy of the proposed filter structure, the Bode plot of transfer function with different loads is plotted in FIG. 7. It is clearly shown that the filter gain is 0 dB at 390 kHz and the filter will pass 390 kHz, and will attenuate 3^{rd}, 5^{th}, 7^{th}, and other higher order harmonics. Filter attenuation to high order harmonics reduces as the load value increases. When the load connected to the filter becomes larger, quality factor of the proposed high order filter becomes smaller. Therefore, the filter frequency selectivity accuracy will collapse and attenuation to high order harmonics will also gradually lose effect. In practical design, PCB traces, inductors and capacitors inevitably have certain parasitic inductance, which might be helpful to dampen harmonics if considered properly.

### III. Experimental Validation

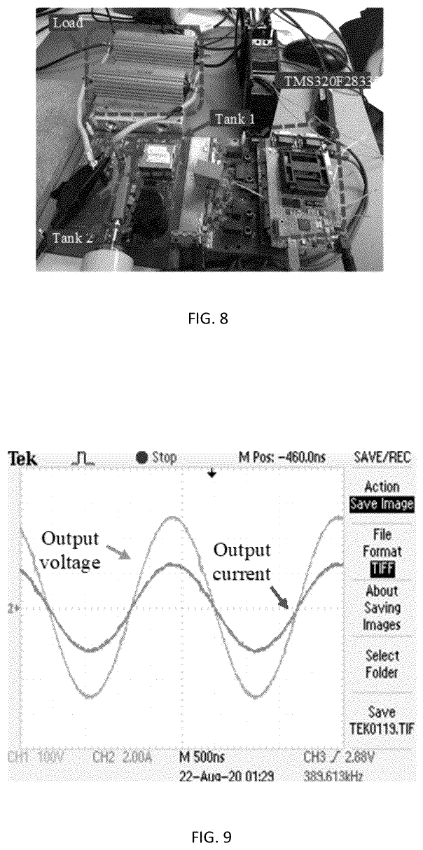

The feasibility of the proposed VHFI is certified by simulation built in Saber and the simulation result is promising. To further examine its practical performance, a 300 W GaN-HEMT based experimental setup, operating at 390 kHz, has been designed and implemented. The PWM signals controlling GaN devices are generated from TMS320F28335 processor and GaN devices are GS66508B from GAN System with 650 V voltage rating. The setup used to conduct experiments is given in FIG. 8 and other components used in this paper, such as inductors and capacitors, are tabulated and listed in Table I.

TABLE I
experiment parameters used in this paper.

| Parameters | Values |
|---|---|
| Power switches Q₁~Q₄ | GS66508B |
| DSP processor | TMS320F28335 |
| Capacitor C₁~C₄ | 1644 pF, 196 pF, 141 pF, 154 pF |
| Inductor L₁~L₄ | 101 uH, 94 uH, 47 uH, 22 uH |
| Switching frequency fₛ | 390 kHz |
| Load resistor | 60 Ω~310 Ω |

A. Continuous Output for Pure Cutting Mode

Electrosurgery requires several clinical operation modes, such as pure cutting mode, blend cutting mode and coagulation mode, and each operation mode requires different output waveforms. When ESG is operating under pure cutting mode, continuous output voltage and current are required, and different output power setting is decided by surgeons in actual electrosurgery. FIG. 9 demonstrates one experimental result for pure cutting mode when 100Ω load is connected to VHFI. It can be seen that the output power is around 300 W and efficiency is 87.10% according to the experiment recordings.

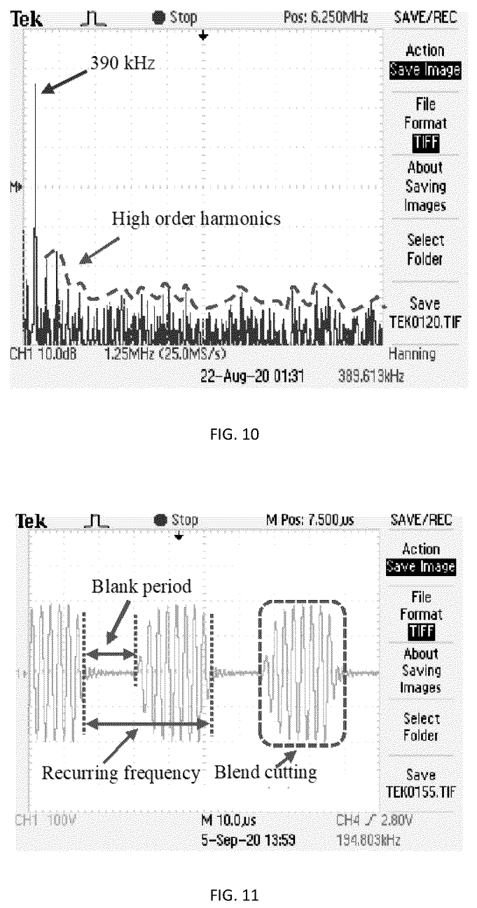

Next, an FFT analysis for output voltage is executed and the frequency spectrum is shown in FIG. 10. The fundamental frequency, 390 kHz, has the magnitude of 68 dB and maximum magnitude of other high order harmonics is less than 23 dB. The equation used to calculate the THD from oscilloscope FFT analysis is written in (3).

$$\{ \begin{matrix}THD = \sqrt{{\sum }_{n = 3 , 5 , …}^{∞} {10}^{\frac{( dBc ) n}{10}}} \\ ( dBc ) n = Mag ( n ) - Mag ( 1 ) , n = 3 , 5 … ∞\end{matrix} \qquad ( 3 )$$

Where Mag(n) is the magnitude of nₜₕ order harmonics read from FFT analysis and (dBc)ₙ represents the magnitude difference between nₜₕ order harmonics and fundamental frequency. It turns out that the approximated output THD is about 1.01% when load is 100Ω and the output voltage is almost pure sinusoidal with 390 kHz.

B. Recurring Output for Blend Cutting Mode

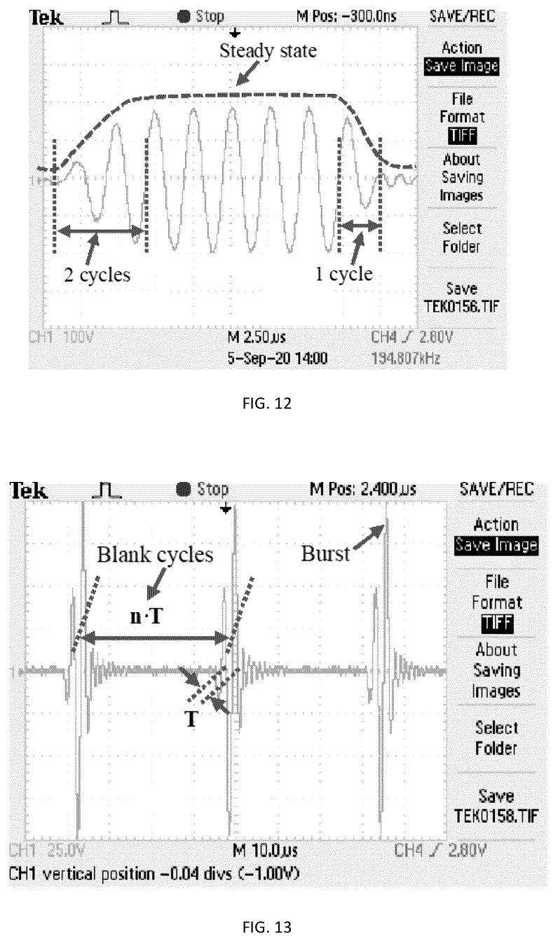

When ESG is operating under blend cutting mode, the output is no longer continuous and blank period appears where output voltage is zero during this time slot. After the blank period, ESG will output sinusoidal waveform again and thus, power is transferred to load in order to elicit clinical blend cutting effect. FIG. 11 examples one typical output waveform for blend cutting operation mode and the maximum output voltage exampled here is about 200 V. The recurring frequency for blank period and blend cutting period will impact the output power and thereby impact the blend cutting effect. More research work is needed in the future to investigate what is the desirable recurring frequency for certain load and what is the duty ratio for blank period. For the case illustration, the blank period in this example is of the same length with blend cutting period and the recurring frequency is set as 27 kHz. The zoomed waveform for blend cutting period from FIG. 11 is plotted in FIG. 12. The output voltage during blend cutting period approaches steady state magnitude within 2 cycles and reduces to very low magnitude within 1 cycle before entering blank period, which showcases the fast-responding capability of proposed VHF. In the meanwhile, it also proves the proposed VHFI can sustain blend cutting mode.

C. Burst Output for Coagulation Mode

When ESG is operating under coagulation mode, sinusoidal burst is required to coagulate human tissues. To get burst output, gate signals triggering GaN devices should be configured correspondingly, so that full bridge can output single discrete square-waveform. And then, the single burst, Vₛ(t), will be reshaped by high order filter into sinusoidal burst which is needed to feed energy into load. FIG. 13 provides a basic demonstration for single burst output and the maximum output voltage is configured as 100 V. The burst strength during blank cycles should be as close as possible to zero and the blank cycle length can be regulated to control the output burst crest factor (“CF”). Crest factor is the ratio between the maximum value of output waveform and the root mean square (“RMS”) of the same output. What is more, CF is a very important indicator to evaluate the waveform extremity and burst output with higher CF indicates less redundant ringing. Blank cycles here are configured as 14 and the approximated crest factor achieved in this paper is about 21. Such high crest factor means the proposed VHFI is able to establish burst output in a very fast manner.

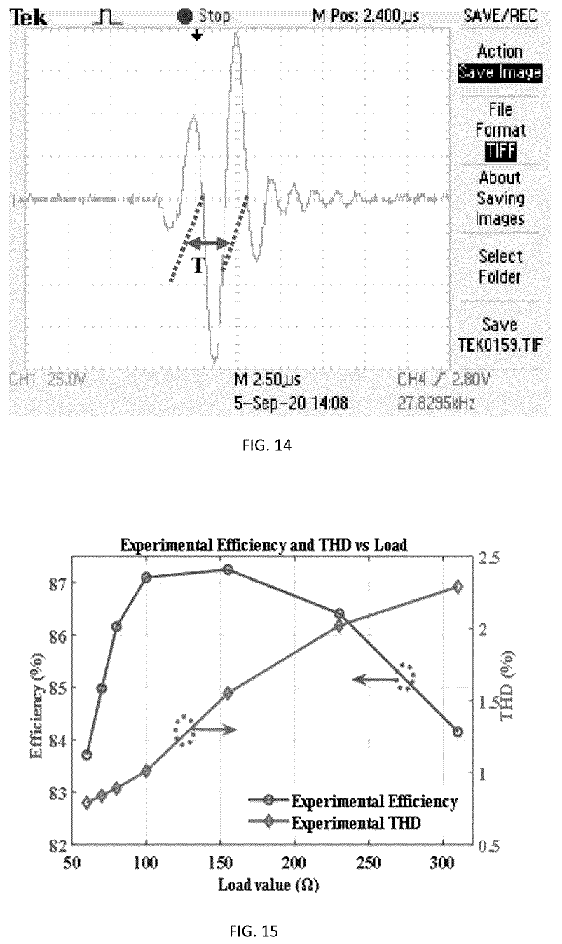

The burst output in FIG. 13 is amplified and shown in FIG. 14. It can be clearly seen that single burst output is established rapidly when the burst is configured, and the output magnitude will decay to zero quickly if blank period is demanded. It verifies previous analysis again and it also proves that proposed VHFI is compatible to coagulation mode.

D. Capability of Supporting Variable Loads

To check the performance of proposed VHFI in term of supporting variable loads, different experiments are conducted under the continuous operation condition where the output power is always set around 300 W and the load is manually altered from 60Ω to 310Ω. During the experiments, it is found that the output voltage will gradually rise as power fed to load increases. The experimental recordings show that peak-to-peak output voltage reaches 940 V when 300 W is delivered to 310Ω load, which is relatively high compared to other high output frequency inverters.

The experimental efficiency and THD versus different load values are gathered and lineated in FIG. 15, where output power is manually maintained around 300 W. If constant output power should be automatically held without knowing load values, then high-frequency current and voltage sensing plus innovative control algorithm are of necessity in the future research.

FIG. 15 also shows that THD deteriorates as load value increases and THD reaches the highest value at 310Ω, which is consistent with previous analysis. Thanks to the large inductor chosen in this example, THD is low and less than 2.5% within all load range. The price for low THD is low efficiency, where the highest efficiency is less than 87.5%. When a large inductor is adopted at such high-frequency, winding resistance, parasitic capacitance, temperature rise, and core material matter and will significantly reduce efficiency. Besides, when a large inductor is chosen, mismatch between capacitor and inductor increases due to inaccurate tuning or parameter variations, will compromise efficiency further. All these factors add together will make the proposed high order filter lossy and depress the overall efficiency. Hence, advanced magnetic design is of significance for the filter inductor here and also inductor parameters should be optimized between THD, efficiency, cost and capacitor voltage stress, etc., such that the best tradeoff is achieved.

### IV. Conclusions

To deliver high-frequency power to electrosurgery, a high output frequency AC inverter is required. However, class E family, Φ₂ inverter and its variants are not able to tackle variable loads and high output voltage at the same time. And thus, a full-bridge based VHFI prototype is described in this example. Its high output frequency producing mechanism and high order filter design are explained in detail. To check practical feasibility of proposed VHFI, a 300 W GaN-HEMT based experimental platform, switching at 390 kHz, has been fabricated and evaluated. The experiment results demonstrate that VHFI is qualified to simultaneously support variable loads and high voltage for a pure cutting mode. The proposed VHFI possesses very low THD, when it is under continuous cutting mode, and the highest THD is less than 2.5%. In addition, the proposed VHFI can also support recurring output and burst output required by blend cutting and coagulation mode, respectively. Further, the proposed VHFI has extremely fast-responding time which takes only 2 cycles for blend cutting mode reaching steady state, and 1 cycle for output magnitude falling to zero. For coagulation mode, the estimated crest factor of proposed VHFI is quite high compared to that in the art and reaches around 21. To validate the accommodation for variable loads, the VHFI is tested with different loads and the experimental efficiency and THD versus different loads are presented. However, the resulting efficiency curve reveals that one drawback of proposed VHFI is its low efficiency, and the highest efficiency is lower than 87.5%. Therefore, efficiency improvement is one of the key research work for the proposed VHFI in the future. Two loss sources are observed as dominating the low efficiency. One is the switching loss affiliated to GaN devices and the other is the loss originating from inductor winding resistance and core loss. As a result, soft switching technique is one of the promising solutions to tackle switching loss. And optimization for inductor design is another effective method to handle the low efficiency issue and achieve the best tradeoff among resonant capacitor voltage stress, total cost, tuning difficulty, THD, core loss, etc. Thereby, high efficiency and low THD can be gathered together inside the proposed VHFI.

### Example 2: Multi-Resonant-Frequency Filter for an Electrosurgery Inverter

This example presents a multi-resonant-frequency (“MRF”) filter for a high-frequency inverter (“HFI”) used in electrosurgery. The fundamental (sinusoidal) output frequency of the HFI is 390 kHz and is the same as the switching frequency of the HFI. The MRF filter is designed to extract the fundamental frequency of the tri-state bipolar waveform, generated by the HFI operating with phase-shift control. The structure and operation of the MRF filer are outlined. An experimental 300 W GaN-FET-based HFI prototype is developed to validate the feasibility of the proposed MRF filter under closed-loop control.

### I. Introduction

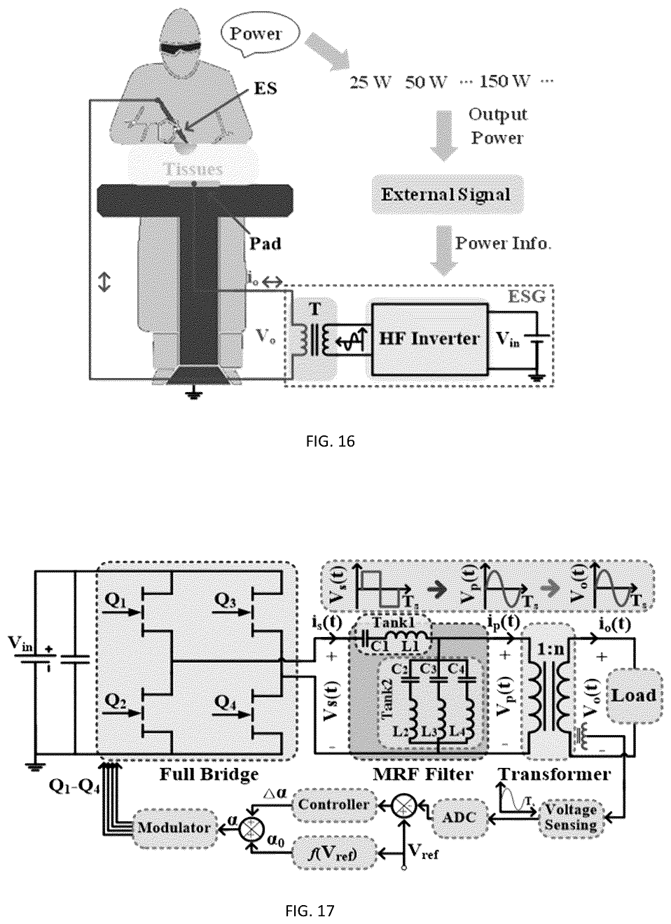

Morton, in 1881, showed that human being's muscle stimulation terminates whenever the applied signal frequency is higher than 100 kHz. Based on that, electrosurgery was first invented in 1926 by William Bovie and the illustration of the monopolar surgery using the electrosurgery generator (“ESG”) is demonstrated in FIG. 16. Modern electrosurgery passes alternating signals with a fundamental frequency above 200 kHz but lower than 5 MHz through the human body to conduct clinical treatment, such as cutting, coagulation, and fulguration, etc. Therefore, inverters with HF outputs are required for electrosurgery.

Resonant inverters (e.g., class E, F, EF₂, Class Φ₂) and their variants feature HF output with reasonable efficiency. However, none of those topologies has been investigated in the area of electrosurgery due to certain limitations, such as load sensitivity, high device voltage stress, and sophisticated tuning processes. Wide-bandgap (WBG) (e.g., SiC, GaN) device-based PWM inverters can operate at HF. However, to generate a fundamental (inverter-sinusoidal-output) frequency of at least 200 kHz, as needed by electrosurgery, an extremely high switching frequency (multi-MHz) is needed that is impractical from efficiency, thermal, and electromagnetic interference standpoints. Selective harmonic elimination (“SHE”) was pursued in line-frequency-commutated converters decades back owing to very-slow thyristor and triacs. However, introduction of notches is still required for extracting the fundamental-frequency output at an acceptable THD and hence application of SHE will still require an electrosurgery HF inverter to operate at frequencies that are multiples of 200 kHz yielding similar challenges mentioned above.

The existing literature on inverters for electrosurgery either outline inverters that generate square-wave output with a much higher switching frequency that yields higher switching loss and more importantly potentially tissue-damaging super-harmonics or soft-switched inverters that reduce the switching loss but can only support very narrow load range (to achieve soft switching) and further, the load range bounds are also well short of the ones needed for supporting electrosurgery.

For this reason, the MRF filter is proposed to tackle the HF inverter challenges faced in the electrosurgery area. This example section introduces the MRF filter structure, HF generation mechanism, and its actual inductance-based transfer function. Based on that, experimental results showing closed-loop performances are provided to justify the feasibility of the proposed MRF filter.

### II. HF Output Generation and MRF

A. HF Output Generation Mechanism

The proposed HFI topology together with its closed-loop control diagram is shown in FIG. 17. DC input of the HFI is denoted by Vᵢₙ and GaN FETs, switching at 390 kHz under phase shift, are represented by Q₁₋₄. When diagonal devices Q₁ and Q₄ (or Q₂ and Q₃) are turned on at the same time, full-bridge output equals Vᵢₙ (or −Vᵢₙ), otherwise, the output is zero. The ideal output of the HFI, Vₛ(t), is delineated as a bipolar square waveform of 390 kHz, which is expressed using a Fourier series as follows:

$${V}_{s} ( t ) = \frac{4 {V}_{i n}}{π} \cdot \frac{1}{n} \cdot cos ( α ) \cdot {\sum }_{n = 1 , 3 , 5 , …∞}^{∞} sin ( 2 π {nf}_{s} t ) \qquad ( 1 )$$

where n and α are the order of the harmonics and the phase shift angle, respectively. The MRF filter practically suppresses the odd harmonics in Vₛ(t) and only the fundamental frequency component of Vₛ(t), appears in the transformer primary side and delivers energy to the load via the high-frequency step-up transformer. Consequently, the output frequency of the HFI is the same as the switching frequency of the GaN-based HFI.

B. MRF Filter

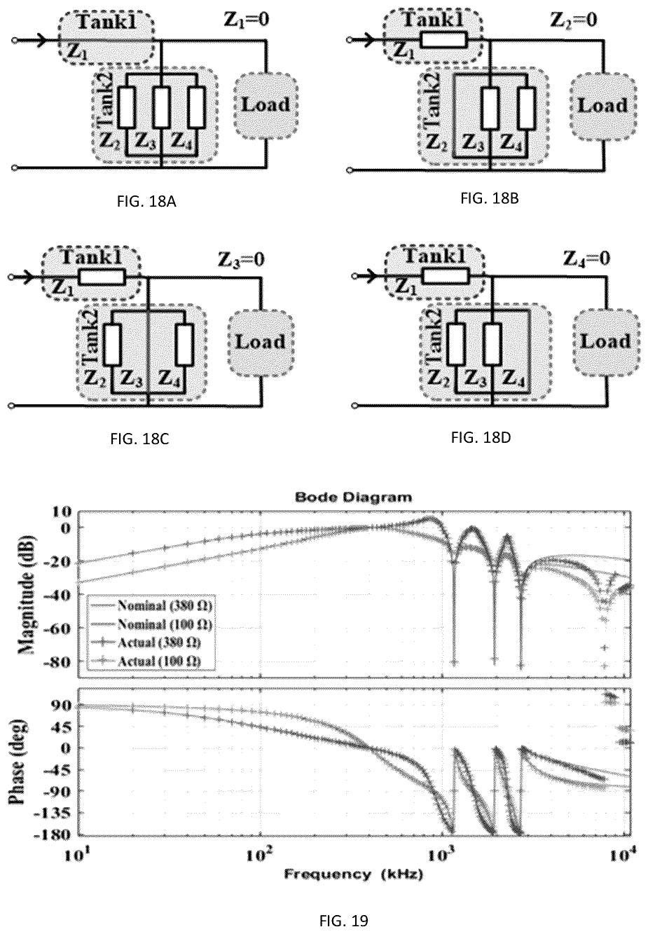

The proposed MRF filter, as shown in FIG. 17, consists of two resonant tanks. Resonant tank-1 is tuned to resonate at 390 kHz, while tank-2 resonates at 3^{rd}, 5^{th}, and 7^{th} order harmonics. An infinite number of resonant branches in tank-2 is theoretically required to eliminate all odd frequencies in Vₛ(t), as shown in (1). However, it turns out 3 resonant branches in tank-2 are good enough to achieve satisfied THD in practice without the need for 9 th or higher resonant branches. Owing to this special structure and combination, the MRF represents different impedances to the fundamental and higher-order harmonic components, which is illustrated in FIGS. 18(a)-18(d). Tank-1 resonates at 390 kHz, and thus, presents zero impedance at ƒₛ, as manifested in FIG. 18(a). As shown in FIGS. 18(b)-18(d), tank-2 yields zero impedances for the 3^{rd}, 5^{th}, and 7^{th} harmonics.

For the MRF, tank-1 L1 is based on CoilCraft AGP4233 series inductor. For tank-2, SER2211 and SER1390 series inductors from CoilCraft are used for L2-L4. Finally, all resonant capacitors are based on C0G dielectric. The MRF transfer function for a given load (R) is determined to be the following:

$${TF}_{MRF} ( s ) = \frac{{V}_{p} ( t )}{{V}_{S} ( t )} = \frac{{Z}_{tank 2} ( s ) ‖ R}{\{ {Z}_{tank 2} ( s ) ‖ R ) \} + {Z}_{tank 1} ( s )} where \qquad ( 2 )$$

$${Z}_{tank 1} ( s ) = s \cdot {L}_{1} + \frac{1}{{sC}_{1}}$$

$${Z}_{tank 2} ( s ) = \frac{1}{\frac{1}{s \cdot {L}_{{2}^{+ 1} {/}_{s} \cdot {C}_{2}}} + \frac{1}{s \cdot {L}_{{3}^{+ 1} {/}_{s} \cdot {C}_{3}}} + \frac{1}{s \cdot {L}_{{4}^{+ 1} {/}_{s} \cdot {C}_{4}}}}$$

and is plotted in FIG. 19. The nominal plot shown in FIG. 19 represents (2) based on the nominal tank parameters. The other plot is based on actual (practical) tank parameters and shows some modest deviation primarily at high frequency (i.e., beyond the fundamental). This deviation is mainly attributed to L₁, which attains progressively higher values with increasing excitation frequency towards the self-resonant frequency of L₁. Tank-2 LC parameters are more stable over the frequency range considered in this paper.

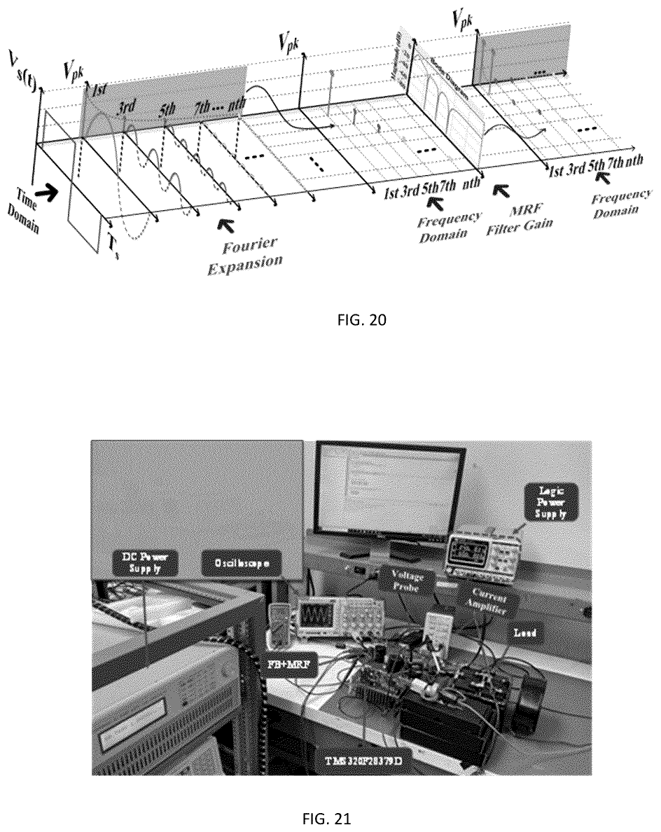

Overall, and as shown in FIG. 19, the two Bode plots both yield 0 dB at 390 kHz and possess small gain at both high-order harmonics (e.g. 3^{rd}, 5^{th}, and 7^{th}, etc.) and frequencies lower than 100 kHz, which distinguishes the MRF filter from conventional low pass filters, such as LCL, and LLCL, etc. Because of such gain characterization, an output of low THD and suppression of frequencies causing muscle stimulation are achieved, simultaneously. However, two Bode plots differ from each other when the frequency exceeds multi-MHz and the actual inductance-based Bode plot imposes higher attenuation. Given the special Bode plot property of the MRF, frequency spectrum components contained in Vₛ(t) are treated differently, as shown in FIG. 20. It is apparent that, the fundamental frequency component of the HFI is passed to the load without reduction whereas other higher harmonics are blocked. Consequently, the bipolar square waveform is shaped into a sinusoidal output, and the transformer primary side voltage, Vₚ(t), is determined as follows:

$${V}_{p} ( t ) = \frac{4 {V}_{in}}{π} \cdot cos ( α ) \cdot sin ( 2 π {f}_{s} t ) \qquad ( 3 )$$

### III. Experimental Results

A 300 W GaN-based HFI hardware prototype, as shown in FIG. 21, is designed, fabricated, and tested to validate the proposed MRF filter. The pulse-width modulation (“PWM”) signal is generated by a TMS320F28379D dual-core DSP controller and the GaN devices are GS66508B from GaN System. The detailed hardware parameters are tabulated and listed in Table I.

TABLE I
Experimental parameters used in this example.

| Parameters | Values |
|---|---|
| Capacitor C₁~C₄ | 3543 pF, 196 pF, 141 pF, 154 pF |
| Inductor L₁~L₄ | 47 uH, 94 uH, 47 uH, 22 uH |
| Switching frequency fₛ | 390 kHz |
| Transformer turn ratio | 2.396 |
| PI parameters | Kₚ = 4.0, Kᵢ = 8.0 |
| Load resistor | LPS 600 series |

A. Output Regulation and THD

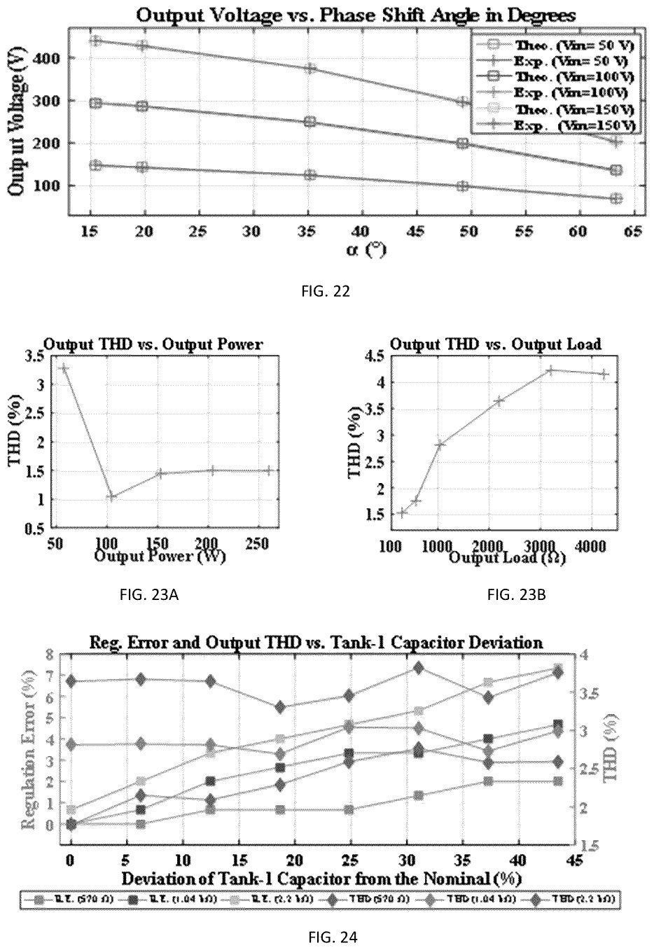

As indicated by (3), the phase shift angle α regulates the transformer's primary side voltage, and thus, secondary-side voltage, Vₒ(t). The theoretical output voltage and experimental measurements are compared and plotted in FIG. 22 when the DC input is set as 50 V, 100 V, and 150 V. The comparison result indicates that the hardware measurements match the theoretical calculation with minimal errors and justify the controllability of a on Vo(t). The output voltage THD for closed-loop control is plotted in FIG. 23 versus different output powers and various output load resistances. It is noted that output voltage THD is quite small and the maximum THD for HFI is 3.28% and 4.23% in FIGS. 23A and 23B, respectively. Furthermore, output THD remains small, as shown in FIG. 24, even when the tank-1 capacitor deviates from its nominal value. It is worth mentioning that output voltage regulation errors slightly deteriorate as the deviation and load resistance increase.

B. Closed-loop Transient Results

To showcase the controllability of the proposed MRF, the closed-loop control diagram for output voltage regulation is given in FIG. 17. The combination of feedforward and proportional-integral (PI) control strategy is employed. The PI parameters are listed in Table I and the feedforward function to calculate α₀ is noted as follows.

$${α}_{0} = f ( {V}_{ref} ) = \frac{180}{π} \cdot {cos}^{- 1} ( \frac{π \cdot {V}_{ref}}{4 \cdot n \cdot {V}_{in}} ) \qquad ( 4 )$$

The feedforward block provides an initial phase shift angle α₀ and the PI controller output Δα compensates for the remaining regulation errors by adjusting the phase shift angle α between gate signals of diagonal switch pairs (Q₁ and Q₄ or Q₂ and Q₃). Based on the control diagram, output start-up transient from zero-state to steady-state is plotted in FIGS. 10A-B. The HFI starts at T₀ and reaches the V_{ref} in 2 switching cycles. Furthermore, output dynamics are provided in FIGS. 11A-B for a step-change in the load. The output voltage slightly reduces at T₁ and then quickly returns to steady-state in 3 cycles. The output has a small overshoot at T₂ and then settles to steady-state in 3 switching cycles.

### IV. Conclusions

This example outlines a MRF filter of a full-bridge-based HFI for electrosurgery. It enables the fundamental output and switching frequency of the HFI to be the same at 390 kHz without requiring complex, or very-high-frequency PWM or expensive solutions. The HF output generation mechanism is explained and the MRF structure and its transfer function are provided as well. A 300 W experimental GaN-based HFI is developed and tested. The experiment results show that the HFI has the capability of regulating output voltage via phase-shift angle. Meanwhile, the HFI also supports a wide range of loads with low output voltage THD under various output powers and load conditions. Furthermore, output THD and regulation errors remain small as the tank-1 capacitor deviates from its nominal value. However, the output THD and current quality slightly deteriorate at light load and high load resistance, which can be improved by properly optimizing the transformer turn ratio. Finally, the feedforward and PI-based control ensures that the transient performance of the HFI is found to be satisfactory despite high order of the HFI, as validated by the experimental results when using transient and load step changes.

### Example 3: Reduced Collateral Tissue Damage Using Thermal-Feedback-Based Power Adaptation of an Electrosurgery Inverter

Well-selected power with accurate delivery is of importance in electrosurgery to generate proper temperature at the cutting site, and thus, reduce undesired collateral tissue damages. Conventional electrosurgery generator (“ESG”) targets tracking a preset power, manually set by surgeons per their experience before the surgery, with high accurate delivery. It is possible that this fixed power setting is not at the optimal point and thus, increases the possibility of added-collateral biomedical tissue damage. To eliminate the potential negative impact of the fixed and ill-suited power setting, a real-time feedback control scheme is outlined in this paper to adjust the preset power of the ESG to create an adaptive power reference, which is then tracked using an experimental high-frequency inverter (“HFI”) that enables electrosurgery with a fundamental (sinusoidal) output frequency of 390 kHz. Subsequently, experiments using the GaN-based HFI are carried out to demonstrate the efficacy of the new variable-power approach over the conventional fixed power approach in terms of collateral tissue damage reduction.

### I. Introduction

Electrosurgery has been used for around a century. It applies HF voltage across conductive biomedical tissue along with the current dictated by the tissue impedance to elicit a clinical effect, such as cutting, coagulation, etc. The mechanism of bio-tissue incision or removal roots in the Joule energy converted from the applied electrical active power. The tissue liquid is rapidly heated up by the energy to the point of vaporization and then the tissue disperses in the form of smoke and stream. The cutting effect on a certain tissue is tightly related to the total energy delivered to it. As is known, energy is the integration of power and time, therefore, both cutting speed and cutting power impose an impact on the final cutting effect. Mismatched cutting speeding or poorly-regulated cutting power either is not able to generate desired clinical effect or may cause undesired added tissue damage, such as charring, thermal spread, dragging and so on.

ESG cutting speed is exclusively controlled by surgeons according to their clinical experience and expertise. Therefore, traditional ESG aims at delivering the power, manually set by surgeons before the surgery, as accurately as possible regardless of the tissue variation. Conventionally, this power is maintained the same during the entire electrosurgery until it is manually updated by the surgeon. As a result, the interruption of the time-sensitive surgery inevitably occurs. Furthermore, others have shown that different ESG power settings have an impact on cutting effects and thermal response, but these experiments did not incorporate thermal-feedback-based real-time power adaptation. Therefore, there is a chance that the power is not optimally orderly set and leads to increased tissue damage or undesired cutting effects. The literature either focuses on improving the power tracking accuracy or pursues a prompt response to the power setting. In one example, infrared sensing is employed to record liver surface-temperature distribution under a single power setting, and it turns out that a small radial region surrounding the ES has the highest temperature during cutting. Moreover, it is reported that thermography-based sensing can be utilized to identify solar cell aging, hotspots, and partial shading faults. However, thermal sensors are employed in existing work simply for temperature measurement. None of the known art links thermal feedback with real-time power adaptation for electrosurgery. As such, the ill-suited power setting issue persists and its resolution via power adaptation is of interest for an ESG.

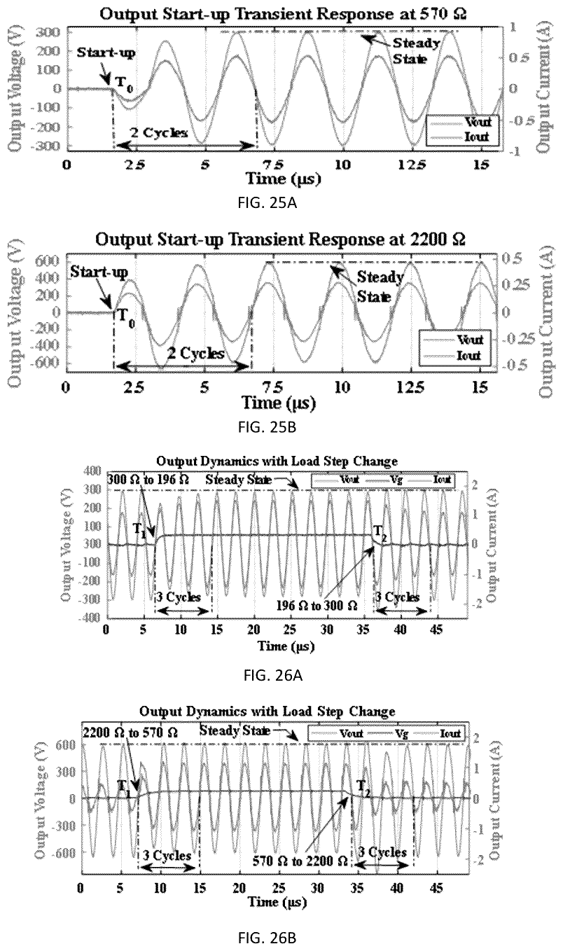

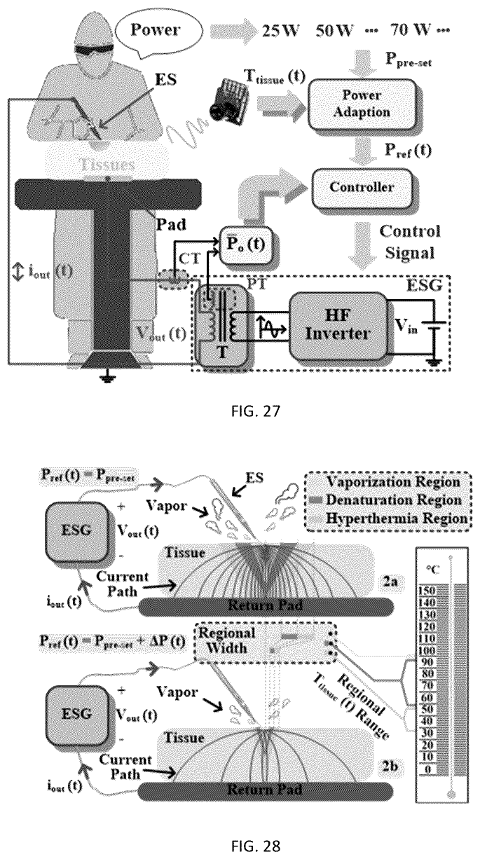

To tackle the ill-suited power setting challenge and reduce collateral tissue damage, a novel real-time thermal-feedback-based closed-loop control scheme is pursued, as illustrated in FIG. 27, in which a tissue surface temperature measurement from the electrosurgery incision site is used to adjust the ESG preset power to create an adaptive power reference, which is then tracked by a power-controller in an experimental HFI. Section II outlines how an ill-suited ESG setting can cause added tissue damage, while Section III outlines the thermal-feedback-based power adaptation scheme. Section IV provides experimental results, while Section V captures the conclusions.

### II. Illustration of Impact of Ill-Suited Power Setting

More than 70% weight of soft tissue comprises water and the tissue is removed when the applied energy vaporizes the water at 100° C. Tissue vaporization is accompanied by another phenomenon, namely, tissue denaturation when tissue cell temperature is between 60° C. and 100° C. Physical properties of tissues among individuals exhibit differences associated with gender, age, size, etc., and those differences escalate the extent of tissue denaturation when the constant power is indiscriminately applied. Tissue denaturation occurs due to undesired thermal spread, and it should be minimized by adjustment in power reference P_{ref}(t), by modulating it with ΔP(t) around the Pₚᵣₑ₋ₛₑₜ, when cutting speeding is fixed.

This point is illustrated in FIG. 28 with two scenarios. In the first scenario, illustrated in FIG. 2, segment 2a, P_{ref}(t) is not varied but kept fixed at P_{ref}=Pₚᵣₑ₋ₛₑₜ. Because ΔP(t)=0 for this scenario, if Pₚᵣₑ₋ₛₑₜ is ill-suited and say higher than what is really needed for safe electrosurgery, then, in the absence of power adaptation, more energy is delivered in a time interval. Consequently, the temperature of a larger volume of tissues reaches the vaporization and denaturation range, dictated by the overall specific heat capacity. As a result, unnecessary tissue removal and denaturation occur, leading to collateral damage even in the presence of a power-control loop since the latter will only ensure that P̅ₒ(t)=P_{ref}(t)=Pₚᵣₑ₋ₛₑₜ. In the second scenario, as illustrated in FIG. 2, segment 2b, P_{ref}(t) has the ability to vary with time due to the power adaptation ΔP(t) that is guided by the thermal feedback from the incision site. As such, P_{ref}(t)=Pₚᵣₑ₋ₛₑₜ+ΔP(t), which ensures that a desired P_{ref}(t) is obtained to minimize the collateral tissue damage.

### III. Power Control Schemes: Constant Vs Adaptive

The full-bridge and bandpass filter-based HFI was initially introduced in Example 1 above and detailed in Example 2 above and it is redrawn in FIG. 29 with the integration of thermal-feedback-based power adaptation.

The fundamental (sinusoidal) output frequency and switching frequency of the HFI are set to be 390 kHz. Referring to FIG. 29, the ideal sensed output voltage of the above-mentioned HFI (Vₒ(t)) is captured:

$${V}_{o} ( t ) = \frac{4 {V}_{in}}{π} \cdot {n}_{t} \cdot cos ( α ) \cdot sin ( 2 π {f}_{s} t ) \cdot {v}_{scaling} \qquad ( 1 )$$

where Vᵢₙ and ƒₛ are the input voltage and the full-bridge switching frequency, respectively. nₜ is the transformer turn ratio, α is the phase shift angle between the diagonal switches in the full-bridge, and v_{scaling} represents the voltage-sensor scaling. The corresponding output power Pₒ(t) for a linear load, with a load angle of θ, is given by:

$${P}_{o} ( t ) = \frac{{V}_{o \_ pk} \cdot {I}_{o \_ pk}}{2} \cdot ( cos ( θ ) - cos ( 4 π {f}_{s} t + θ ) ) \cdot {ρ}_{scaling} \qquad ( 2 )$$

where V_{o_pk} is the peak of Vₒ(t) and I_{o_pk} is the peak of iₒ(t), the scaled output current of the HFI. ρ_{scaling} is the coefficient that maps sensed Vₒ(t) and iₒ(t) back to actual output power. As seen, the first item of the Pₒ(t) is a constant component, which is the average power (P̅ₒ(t)) over an HFI fundamental-output cycle while the second term represents an ac perturbation term that evolves at double the fundamental-output frequency. This example aims to properly control the output voltage and thus current, such that P̅ₒ(t) is well-regulated and follows the adaptive power reference to reduce collateral tissue damage.

A. Constant Power Control

The control block diagram in FIG. 29 achieves constant power control by setting ΔP(t)=0 and yielding P_{ref} (t)=Pₚᵣₑ₋ₛₑₜ.

The constant power reference is then compared with feedback P̅ₒ(t) and the error is fed to a proportional-integral-based (PI) controller, which ensures the realization of the desired power. In actual electrosurgery, surgeons select Pₚᵣₑ₋ₛₑₜ according to their clinical needs and professional experience. However, owing to physical properties varying among patients, Pₚᵣₑ₋ₛₑₜ may be improperly set, which may cause erroneous incision-site temperature due to a mismatch between the fixed power reference set for the electrosurgery (i.e., P_{ref}(t)=Pₚᵣₑ₋ₛₑₜ) and the P_{ref}(t) that is actually needed. Consequently, undesired cutting effects and collateral tissue damages are elicited, such as thermal spread, carbonization, burns, etc.

B. Thermal-Feedback-Based Power-Adaptation Control

To mitigate the collateral tissue damage due to nonoptimality in Pₚᵣₑ₋ₛₑₜ, the proposed thermal-feedback-based power adaptation control monitors tissue surface temperature using an infrared sensor and feeds it back at a frequency of 8 Hz. Based on the sensed temperature data, the power-adaptation controller feeds in real-time a power adaption term (ΔP(t)):

$$Δ P ( t ) = {P}_{pre - set} \cdot ( \frac{{T}_{nom}}{max ( {T}_{tissue} ( t ) )} - 1 ) \qquad ( 3 )$$

to the P_{ref}(t) yielding the following, as captured in FIG. 29:

P_{ref}(t)=Pₚᵣₑ₋ₛₑₜ+ΔP(t). (4)

In (3). Tₜᵢₛₛᵤₑ(t) is the tissue surface temperature, max(Tₜᵢₛₛᵤₑ(t)) is the maximum of Tₜᵢₛₛᵤₑ(t), and Tₙₒₘ is the nominal tissue temperature that ensures safe electrosurgery with minimal/no collateral damage. By following (4), any time the max(Tₜᵢₛₛᵤₑ(t)) exceeds Tₙₒₘ, P_{ref}(t) is so adjusted such that the incision-site tissue temperature is brought back close to Tₙₒₘ thereby mitigating collateral tissue damage.

### IV. Experimental Results

A GaN-FET-based hardware prototype, as shown in FIG. 30, is used to examine the cutting effects of the proposed thermal-feedback-based power adaptation control. As shown in the top left corner in FIG. 30, MLX90640 infrared sensor is mounted together with ES such that they move together without relative movement. Consequently, tissue surface temperature around ES is always monitored, and thus the maximum temperature of entire tissue surface is captured during electrosurgery. The sensed thermal data are processed by Raspberry Pi 4 Model B and then transmitted to the DSP controller, which, using this feedback, implements the power adaptation and subsequently, generates the PWM signals for the GaN FETs (i.e., GS66508B from GaN System) of the full-bridge converter of the HFI, as shown in FIG. 29. Fresh pork with 16 millimeters (mm) thickness is placed between the ES and return pad as biomedical load of the HFI. The detailed experimental parameters are summarized in Table I.

TABLE I
Parameters for the experimental hardware setup.

| Parameters | Values | |
|---|---|---|
| Input voltage (Vᵢₙ) | 110 | V |
| Scaling factor | ν_{scaling} = 0.00415, ρ_{scaling} = 154.60 | |
| HFI parameters | The same as in Table I at [00164] | |
| ES moving speed | 5 | mm/s |

| | |
|---|---|
| PI parameters | Kₚ = 0.035, Kᵢ = 160000 |

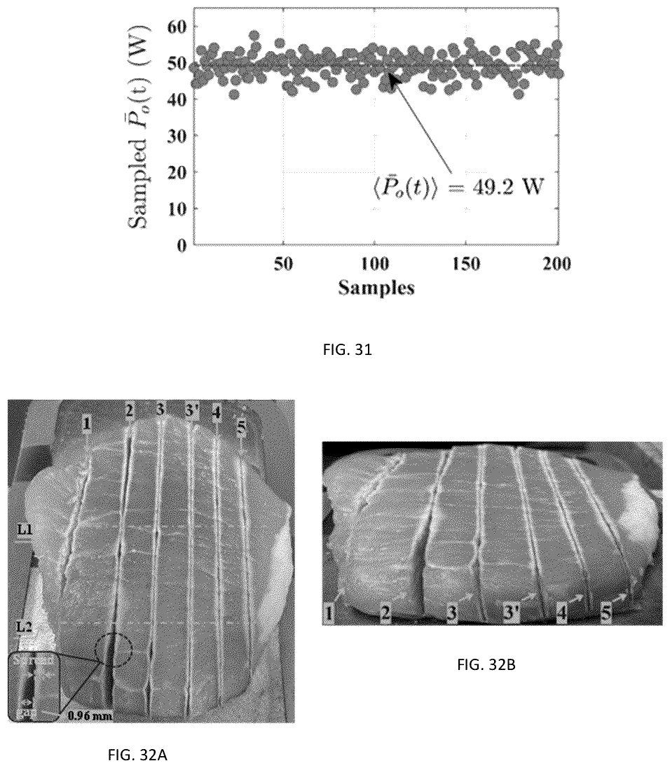

Using this setup, first, the efficacy of HFI operating, under constant power control with P_{ref}(t)=Pₚᵣₑ₋ₛₑₜ=50 W (i.e., ΔP(t)=0) is shown in FIG. 31 with experimental results. This scenario emulates a conventional electrosurgery, where the required output power is preset by the surgeons and the setting remains the same till the surgeons manually change it again.

Next, in FIGS. 32A-B, the scenario when P_{ref}(t)=Pₚᵣₑ₋ₛₑₜ+ΔP(t) is explored. 5 test scenarios are pursued as captured in Table II: test scenarios 1 and 2 correspond to ΔP(t)=0 and Pₚᵣₑ₋ₛₑₜ set at 50 W and 60 W, respectively; test scenarios 3-5 correspond to ΔP(t)≠0 and instead, ΔP(t) is generated for each of the 3 scenarios based on 3 values of nominal temperature (Tₙₒₘ) of 50° C., 55° C., and 65° C., respectively.

TABLE II

| | Test Scenarios | | | | | |
|---|---|---|---|---|---|---|
| | Trials | | | | | |
| Settings | #1 | #2 | #3 | #3′ | #4 | #5 |
| Pₚᵣₑ₋ₛₑₜ | 50 W | 60 W | 60 W | 60 W | 60 W | 60 W |
| ΔP(t) | 0 | 0 | Using (3), | Using (3), | Using (3), | Using (3), |
| | | | Tₙₒₘ = | Tₙₒₘ = | Tₙₒₘ = | Tₙₒₘ = |
| | | | 50° C. | 50° C. | 55° C. | 65° C. |
| P_{ref}(t) | 50 W | 60 W | Using (4) | Using (4) | Using (4) | Using (4) |

TABLE III
Evaluation of experimental cutting effects for the 5 test scenarios.

| | Trials | | | | | |
|---|---|---|---|---|---|---|
| | Test Scenarios | | | | | |
| Metrics | #1 | #2 | #3 | #3′ | #4 | #5 |
| Gap at L1 | 0.66 | 0.06 | 0.10 | 0.04 | 0.09 | 0.06 |
| Gap at L2 | 0.02 | 0.40 | 0.05 | 0.05 | 0.10 | 0.19 |
| Spread at L1 | 0.90 | 0.82 | 0.95 | 0.98 | 1.37 | 1.51 |
| Spread at L2 | 0.02 | 1.99 | 0.99 | 0.90 | 1.16 | 1.70 |
| Gap difference | 0.64 | 0.34 | 0.05 | 0.01 | 0.01 | 0.13 |
| Spread | 0.88 | 1.17 | 0.04 | 0.08 | 0.21 | 0.19 |
| difference | | | | | | |

Note:
All measurements are gauged in millimeters (mm).

The purpose of the test scenarios 1 and 2 is to provide an illustrative approach to the choice of Tₙₒₘ for determining ΔP(t) using (3) (to obtain P_{ref}(t) using (4)) in the last 3 scenarios. As evident in FIGS. 32A-B and Table III, the test scenario 1 corresponding to P_{ref}(t)=Pₚᵣₑ₋ₛₑₜ=50 W yields less cutting gap and thermal spread than test scenario corresponding to P_{ref}(t)=Pₚᵣₑ₋ₛₑₜ=60 W. Based on these 2 tests, an estimated Tₙₒₘ range was conjectured to be between 50° C. and 65° C. guided by the outcomes of test scenarios 1 and 2.

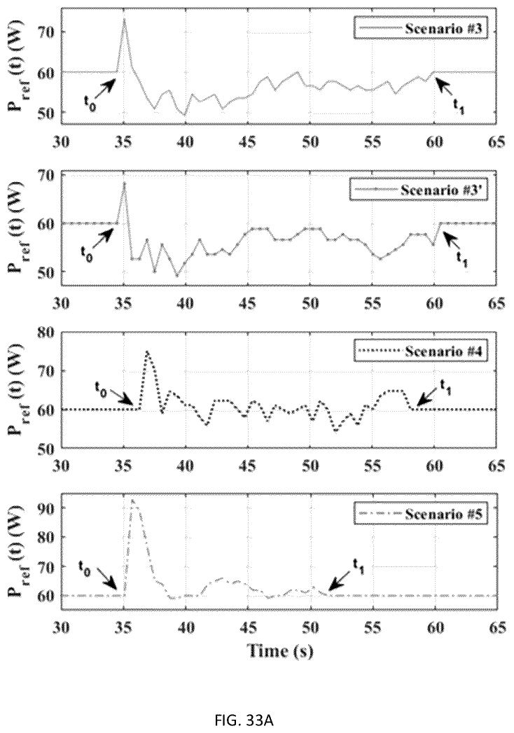

Using that range, the test scenarios 3-5, following Table II, were pursued and the results are captured in Table III. The latter shows that test scenario 3 together with its repeated trial 3′ yields overall the best results and this is evident in FIGS. 32A-B as well. It illustrates that, compared to test scenario 1, which yielded comparable cutting temperature but with a larger temperature ripple, the results are better for test scenario 3 because of the power reference adaptation, as shown in FIG. 33A, which also adjusts the maximum tissue surface temperature accordingly with smaller temperature ripple, as shown in FIG. 33B. The superior result for test scenario 3 over test scenarios 4 and 5, as evident in Table III and FIGS. 33A-B, is attributed to the fact that the Tₙₒₘ for the latter two cases are set to be higher, leading to higher averaged cutting site temperature and thus wider cutting gap and thermal spread. It worth mentioning that the thermal spread repeatability of test scenario 3 are demonstrated by test 3′ and can be extended to other scenarios as well. Furthermore, the cutting gap uniformity with power adaptation control is also repeatable, as verified by Table III.

### V. Conclusions

This example outlines a thermal-feedback-based power adaptation control to reduce the collateral tissue damage for electrosurgery. The impact of the ill-suited power setting is illustrated, and the power adaptation scheme is elaborated. A GaN-based HFI setup is used to examine the cutting effects of constant power and thermal-feedback-based power adaptation control in terms of cutting gap and thermal spread. The experiment results show that the cutting test with a higher constant power features a larger cutting gap and wider thermal spread. Moreover, with a constant power setting, the tissue is not evenly cut with visible cutting gap and thermal spread difference at two-incision locations. With the proposed power adaptation control, the cutting gap and thermal spread are tangibly reduced with the proper choice of nominal temperature. Moreover, it is also found that thermal sensor location and its resolution have an impact on the accuracy of sensing the surface temperature of the tissue and needs careful attention. Further, the gap and thermal difference at different incision sites are found to be reduced compared to results obtained using a fixed power setting. In other words, cutting uniformity with power adaptation is improved in terms of both cutting gap and thermal spread. It is evident that power adaptation in the vicinity of accurate nominal temperature is the key to reduced collateral damage. In practical electrosurgery, this estimate can be obtained more accurately given the repeatability of reliable cutting performance and the availability of a much larger electrosurgery database.

### Example 4: Output Power Computation and Adaptation Strategy of an Electrosurgery Inverter for Reduced Collateral Tissue Damage

This example investigates two ways of output-power computation, namely, sparse- and multi-sampling-based methods, to overcome sampling speed limitation and arcing nonlinearity for electrosurgery. Moreover, an impedance-based power adaptation strategy is explored for reduced collateral tissue damage.

Methods: The efficacy of the proposed power computation and adaptation strategy are experimentally investigated on a gallium-nitride (GaN)-based high-frequency inverter prototype that allows electrosurgery with a 390 kHz output frequency.

Results: The sparse-sampling-based method samples output voltage once and current twice per cycle. The achieved power computing errors over 1000 cycles are 1.43 W, 2.54 W, 4.53 W, and 4.89 W when output power varies between 15 W and 45 W. The multi-sampling-based method requires 28 samples of both outputs, and the corresponding errors are 0.02 W, 0.86 W, 1.86 W, and 3.09 W. The collateral tissue damage gauged by average thermal spread is 0.86 mm, 0.43 mm, 1.11 mm, and 0.36 mm for the impedance-based power adaptation against 1.49 mm for conventional electrosurgery.

Conclusion: Both power-computation approaches break sampling speed limitations and calculate output power with small errors. However, with arcing nonlinearity presence, the multi-sampling-based method yields better accuracy. The impedance-based power adaptation reduces thermal spreads and diminishes sensor count and cost. Significance: This example exemplifies two novel power-computation options using low-end industrial-scale processors for biomedical research involving high-frequency and nonlinearly distorted outputs. Additionally, this work is the first to present the original impedance-based power adaptation strategy for reduced collateral damage and it may motivate further interdisciplinary research towards collateral-damage-less electrosurgery.

### I. Introduction

Biomedical tissue is made of numerous cells and cellular membranes introduce capacitance to tissue, therefore, the current path inside the tissue is frequency-dependent. Capacitance blocks DC current and presents a high impedance to low-frequency AC current. Hence, the resultant current path mainly surrounds cells through extracellular liquids, as shown in FIG. 34A. In contrast, AC current of high frequency passes through both extracellular and intracellular liquid, as pictured in FIG. 34B. A high-frequency current of sufficient high amplitude, flowing through the cell body, can generate enough Joule heating and raise intracellular liquid temperature to the vaporization point. The concomitant volume expansion of the volatilized liquids gives rise to microscopic cell ruptures, and collectively, produces macroscopical tissue-cutting effects. Besides, muscle and nerve stimulation or pain ends when the applied electrical signal oscillates more than 100 thousand times per second. Grounded on those principles, William Bovie pioneered and devised the first electrosurgery device of real sense about 100 years ago. After that, continuous efforts from researchers promote the development of electrosurgery and enhance the scientific understanding of human society on it. Nowadays, the pursuit of reduced collateral tissue damage becomes one of the critical focuses of modern electrosurgery.

The tissue damage during electrosurgery is tightly related to electrical energy applied to the target tissue. Properly delivered energy not only minimizes additional tissue damage but also shortens the time required for post-surgery recovery. In contrast, inappropriately transmitted energy enhances undesired tissue damage, and increases safety concerns as well. As is well known, electrical energy is the integration of instantaneous power over the period that power from the electrosurgery generator (“ESG”) is activated. The cutting speed solely determines the time interval spent over a certain length when the electrode moves along the tissue surface trace during electrosurgery. Consequently, both applied power and cutting speed (or time interval of power activated) are theoretically supposed to play an important role in tissue damage during actual electrosurgery.

Others have explored thermal damage induced by fixed power with different cutting speeds. The experimental results validate the significant impact of cutting speed on electrocuting damage and emphasize the importance of cutting speed control. Moreover, others have experimentally demonstrated that various activated periods of power lead to different surgical damage even with the same power setting. Instead of varying cutting speed or power activation period, others vary applied power and show the important impact of discrete power settings on the overall cutting performance. Beyond that, others have elaborated on the generation mechanism of collateral damage due to an ill-suited power setting. Based on the foregoing, it can be concluded that cutting speed (or power activated interval) and power setting are experimentally proven to be critical for electrosurgery and should be properly controlled to reduce unwanted collateral tissue damage.

In actual electrosurgery, the cutting speed or time interval of power activation is at the surgeon's sole discretion, except that the electrosurgery is autonomously implemented by a robotic arm. In such a case, the cutting speed or power-activated time interval should be controlled by embedded servo motors inside the robotic arm with very high precision and sensitivity. Nonetheless, in either case, both cutting speed and power activation time interval are externally controlled and none of them is regulated by the ESG itself. The focus of this example centers on the power adaptation of a high-frequency inverter that enables electrosurgical trials, rather than autonomous robotic electrosurgery. Therefore, autonomous control on cutting speed or regulation of power activation time interval is not covered herein.

For conventional electrosurgery, the applied power is exclusively determined and manually entered into ESGs by a surgeon before surgery is initiated. The value of selected power is primarily decided based on surgeons' cumulative clinical experiences. There is a lack of professional procedures indicating how to quantitatively update the power setting when target tissue changes either due to tissue type or physical property variation, etc. The practical physical and electrical properties of tissue differ when it comes from distinct individuals or even from the same individual and the same tissue type but separate locations, etc. Under such circumstances, the optimal power choice, ideally speaking, should be so adjusted such that the induced collateral damage is minimized as much as possible.

Unfortunately, the power setting is usually maintained the same during conventional electrosurgery until further updates are reimported by surgeons when appreciable undesired electrosurgical effects or collateral damages have been irreversibly generated and observed. Those undesirable effects or additional damage prolong the post-surgery rehabilitation duration and should be avoided as far as possible through timely power adaptation. In practice, it is challenging for surgeons to entirely avoid power setting nonoptimality and thus, added collateral damage because they can hardly precisely and promptly identify tissue property discrepancies or variations.

Even if surgeons are hypothetically able to distinguish tissue's physical and electrical property fluctuations, it also takes them some time to halt the electrosurgery, manually reload the power setting into ESG and then reinitiate the surgical procedures again. The total time duration for time-sensitive electrosurgery is elongated as the alteration frequency of such processes climbs. The stretched clinical duration increases the risk of clinical failure or might lead to other serious consequences. Given that, there should be a viable tradeoff between the increasing power modification frequency for reduced collateral tissue damage and the decreasing power modification frequency for shortened electrosurgical time consumption.

The majority of existing literature tracks the manually entered power setting with high accuracy and rapid response. As a result, no real-time power adaptation is adopted in traditional ESG, and surgeons solely take control of the power setting. In contrast to that, a thermal-feedback-based power adaptation that can autonomously modify this power setting has been detailed. Electrocuting traces, conducted on fresh pork using such power adaptation strategy, show superiority over those cut by conventional fixed power in terms of thermal spread and cutting gap uniformity.

However, notwithstanding the apparent merits mentioned above, drawbacks exist for the former thermal-based power adaptation method. A considerable amount of smoke occurs during electrosurgery, and they suffuse between the thermal sensor and tissue, imposing a negative influence on temperature measurement accuracy. Either an advanced and complex thermal sensing compensation algorithm or a costly smoke evacuation pencil is needed to remove the adverse impact of the smoke on tissue surface temperature measurement.

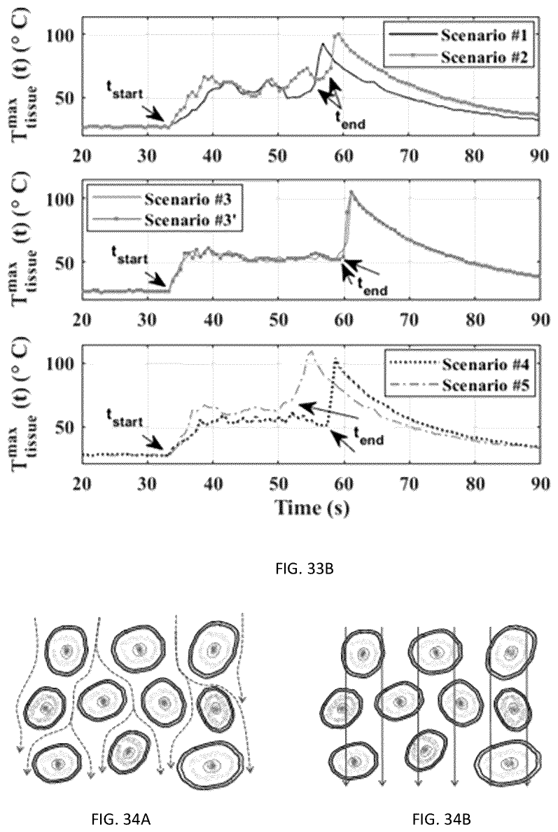

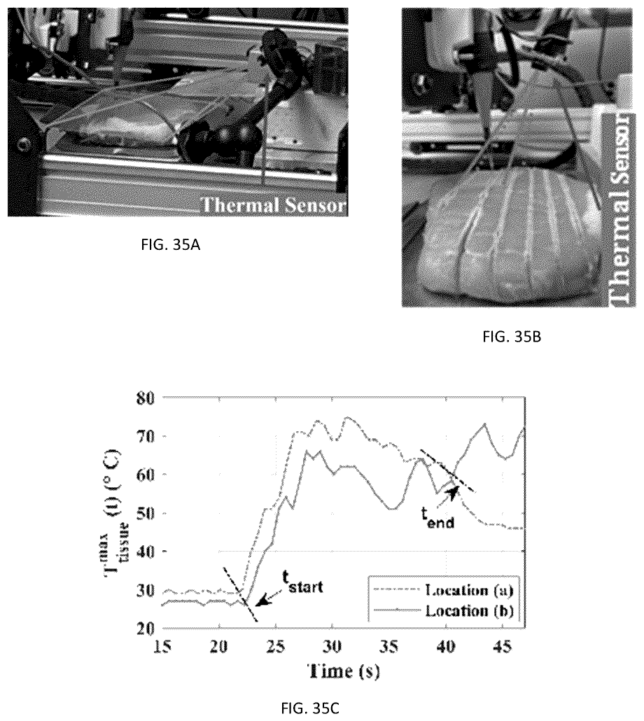

Besides, the thermal sensor mounting location also plays an important role in thermal measurement accuracy, as shown in FIG. 35. In FIG. 35A, the thermal sensor is installed on an external holder such that the entire tissue surface is located within the field view of the sensor. By doing so, the whole tissue surface temperature is measured in each sensing frame, rather than a limited region. In contrast, the thermal sensor in FIG. 35B is mounted together with the electrode such that they move together without relative movement during electrocuting. As a result, only the tissue surface temperature, surrounding the electrode tip, is monitored without or with minimal variation of sensing angle and sensing distance between the electrode tip and the thermal sensor. To showcase the temperature measurement difference for two mounting locations, electrocuting trials are conducted on fresh pork under identical experimental conditions. The measured maximum tissue surface temperature is plotted in FIG. 35C, and significant differences exist between the two sensor-mounting locations, which validates the importance of the thermal installation location.

Furthermore, the thermal sensor resolution also matters and imposes an impact on measurement granularity and precision, etc. Higher resolution yields more accurate thermal sensing while, on the other hand, higher resolution also indicates larger thermal data size, heavier data processing burden, and probably higher sensor cost. Finally, it is worth mentioning that the refresh rate of thermal sensors can hardly go beyond 100 Hz. This significantly limits the application of thermal-feedback-based power adaptation in the instance that is highly sensitive to power settings and requires ultrafast power adjustment.

Considering the limitations mentioned above for thermal-based power adaptation, the impedance-based ultrafast power adaptation is proposed in Section II-D to serve as one promising substitution. Compared to the conventional constant power scenario, the proposed power adaptation method reduces collateral tissue damage. Meanwhile, it eliminates the limitations linked to the thermal-based tactic with additional benefits of reduced sensor count, shrunken budget and communication requirement, etc. The efficacy of this novel method is examined on the full-bridge-based high-frequency inverter (HFI) that is introduced in Example 2 above with a fundamental (sinusoidal) output frequency of 390 kHz.

### II. Methods

A. Sparse-Sampling-Based Power Calculation

It is a practical challenge to sample and precisely reconstruct signals of hundreds of kilohertz without a multi-MHz analog-to-digital converter (ADC) sampling rate. Others have sampled this kind of high-frequency signal at 50 MHz using Xilinx field-programmable gate array (“FPGA”), rather than a low-cost industrial-scale digital signal processor (“DSP”). A high ADC sampling rate is avoided by others for average output power computation. However, the output power is indirectly calculated from the input side of the high-frequency inverter with the assumption of a lossless switch network, rather than directly computed from the output (load) side. The practical switches are lossy, especially switched at high frequency. Consequently, the feasibility of this method only works to some extent and needs more examination for diverse situations. Given that the majority of commercial low-end DSPs possess only a few MHz sampling rates, therefore, a power computing algorithm (“PCA”) is provided to compute the mean value of output power over each cycle. The proposed PCA requires only one output voltage sample and two output current samples per cycle, respectively. These three data can also be utilized to estimate the load impedance in a cycle-by-cycle ultrafast manner.

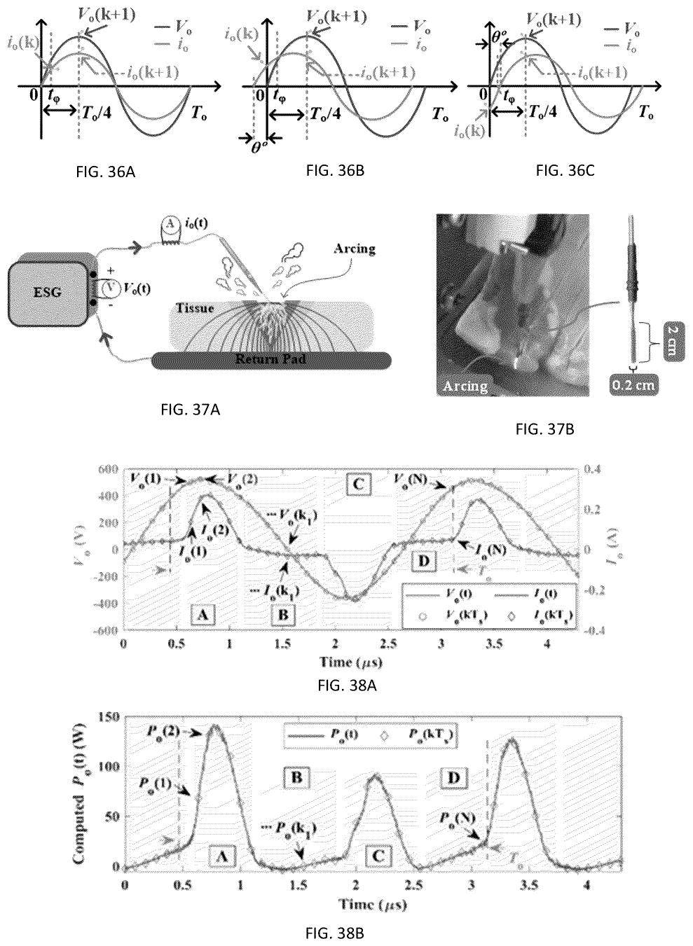

When the ideal sinusoidal voltage is applied to a linear load, the resultant current is also sinusoidal and oscillates at the same frequency. The magnitude and phase of the load current depend on load impedance magnitude and type, such as resistive, capacitive, or inductive. Although the Cole-model indicates the capacitive property of the biotissue, all three linear load characteristics are delineated in FIG. 36A-C for the sake of completeness. The procedures to reconcile the average output power are formulated as follows:
1) Properly configure the sampling timing of the DSP ADC channel for output voltage Vₒ(t) sensing such that it approximately catches the output voltage positive peak at Tₛ/4, noted down as Vₒ(k+1), which can be easily achieved with the HFI in Example 2 above. Although an exact sampling at Tₛ/4 is preferred, there is no need to precisely sample output voltage at Tₛ/4 since sinusoidal signals have minor derivation near the peak. Deviation error is small as long as the sampling point does not seriously deviate from Tₛ/4. Moreover, this kind of error can also be corrected via sensor calibration.
2) Properly configure the DSP ADC current channel such that it samples the output current iₒ(t) twice per switching cycle. The first sample, denoted as iₒ(k), initiates at predetermined t_{φ} that can be any timing between 0 and Tₛ/4 when taking voltage as the reference. The only requirement for iₒ(k) is to avoid overlap with the second sample. The second sample is launched at Tₛ/4 and is noted down as iₒ(k+1). Such a current sampling configuration can also be easily obtained by proper DSP ADC triggering.
3) The quantitative relationship among iₒ(k), iₒ(k+1) and output current magnitude iₒₚₖ(k) is described in (1) if iₒ(k) is sampled at t_{φ} that is equivalent to the electrical angle of φ (in radian). The load impedance angle is denoted as θ (in radian). Then the output current magnitude is calculated as (1a) and the simplified equations for cases where φ=0 and π/6 are written in (1b) and (1c), respectively. Equation (1c) is used by the sparse-sampling-based power calculation in this paper.

$$\{ \begin{matrix}{i}_{opk} ( k ) \cdot sin ( θ + φ ) = {i}_{o} ( k ) \\ {i}_{opk} ( k ) \cdot sin ( θ + \frac{π}{2} ) = {i}_{o} ( k + 1 )\end{matrix} \qquad ( 1 )$$

$${i}_{opk} ( k ) = \sqrt{\frac{{\vert {i}_{o} ( k ) \vert}^{2} + {\vert {i}_{o} ( k + 1 ) \vert}^{2} - 2 \cdot \vert {i}_{o} ( k ) \vert \cdot \vert {i}_{o} ( k + 1 ) \vert \cdot sin ( φ )}{{cos}^{2} ( φ )}} \qquad ( 1 a )$$

$${i}_{opk} ( k ) = \sqrt{{\vert {i}_{o} ( k ) \vert}^{2} + {\vert {i}_{o} ( k + 1 ) \vert}^{2}} \qquad ( 1 b )$$

$${i}_{opk} ( k ) = \sqrt{\frac{4 \cdot ( {\vert {i}_{o} ( k ) \vert}^{2} + {\vert {i}_{o} ( k + 1 ) \vert}^{2} - {i}_{o} ( k ) \vert \cdot {i}_{o} ( k + 1 ) \vert )}{3}} \qquad ( 1 c )$$

4) Then, the load impedance magnitude is estimated as (2):

$$\vert Z \vert = \frac{{V}_{o} ( k + 1 )}{{i}_{opk} ( k )} \qquad ( 2 )$$

5) The impedance angle θ is approximated by (3):

$$θ = {cos}^{- 1} \frac{\vert {i}_{o} ( k + 1 ) \vert}{{i}_{opk} ( k )} \qquad ( 3 )$$

6) The estimated load impedance is captured in (4):

Z=|Z|∠(0) (4)
7) Finally, the continuous instantaneous and average output power per cycle is obtained by multiplying output current and voltage, and then averaged over one cycle. They are dictated in (5) and (6), respectively:

$${P}_{o} ( t ) = \frac{{V}_{opk} \cdot {I}_{opk}}{2} \cdot cos ( θ ) - \frac{{V}_{opk} \cdot {I}_{opk}}{2} \cdot cos ( 4 π {f}_{s} t + θ ) \qquad ( 5 )$$

$${\overline{P}}_{o} ( t ) = \frac{{V}_{opk} \cdot {I}_{opk}}{2} \cdot cos ( θ ) \qquad ( 6 )$$

where Vₒₚₖ equals to Vₒ(k+1) and is the output voltage peak while Iₒₚₖ is the peak of output current and ƒₛ is the output frequency. With the assumption of sinusoidal output voltage and current, the proposed PCA can determine average output power and load impedance in a cycle-by-cycle ultrafast manner with only one voltage sample and two current samples, respectively. Hence, compared with existing literature, the ADC sampling rate requirement is significantly reduced. Furthermore, the PCA only involves simple mathematical operations with low complexity, which enables its real-time implementation in low-end industrial level DSP.

B. Impact of Arcing Presence on Output Current and Power Calculation

Both instantaneous and average power computations using (5) and (6) assume a linear load. This means that the sinusoidal voltage induces sinusoidal current with the same frequency. This assumption is adopted by most of the existing literature, and it is true if no arcing is generated by the applied voltage during electrocuting, which is generally not the case in reality. On the opposite, actual electrosurgery is often accompanied by nonlinear arcing so that the current is no longer sinusoidal. Arcing occurs when the air around the electrode is broken down (ionization of dielectric), as graphically represented in FIG. 37A while FIG. 37B experimentally demonstrates the occurrence of arcing during electrocuting on fresh pork. With the presence of arcing, the lumped currents seen by the electrode have two parts. One part is introduced by the tissue impedance along with the interface impedance between the electrode and tissue. The other part is due to arcing resistance, biotissue impedance, and interface impedance between them. The lumped current is distorted and deviates from a purely sinusoidal shape due to the nonlinearity introduced by the arcing. The degree of lumped current distortion largely depends on the proportion of current flowing in the form of arcing.

With appreciable current distortion and potential voltage asymmetry or distortion, neither output current nor voltage should be assumed as sinusoidal anymore. Because of that, the average output power cannot be calculated by the way described in (6), otherwise, tangible power calculation errors are inevitably induced. On the other hand, it is quite important for an ESG to accurately compute the output power and then precisely track the given reference. For these reasons, a real-time multi-sampling-based power calculating method is set forth herein with the expectation of enhanced power calculating precision. The detailed illustrative explanation for it is presented in the following subsection.

C. Multi-Sampling-Based Power Calculation

The presence of large arcing heavily distorts the output current, thereby, more sampling points are necessary to reflect current characteristics. A graphical representation of output voltage and distorted current is plotted in FIG. 38A with N samples each cycle. In FIG. 38A, the output voltage and current are simultaneously sampled at the same timing as a pair and N pairs in total each cycle. Multiplying voltage and current, the obtained instant power is drawn in FIG. 38B for both original and sampled outputs. The instant power pulsates twice each cycle with nonidentical amplitudes and nonsinusoidal profiles, which highlights the inapplicability of (5) and the hypothesis of sinusoidal outputs again. The instantaneous power and average power for sampled outputs over one cycle are expressed by (7) and (8), respectively.

Pᵢₙₛ(k)=Vₒ(k)·Iₒ(k) (7)

[symbol]

P̅_{avg}(j) [symbol] _{T}_{ₒ}=(Σ_{k=(j-1)·N+1}^{(j-1)·N+N}Pᵢₙₛ(k))/N (8)

where Vₒ(k) and Iₒ(k) symbolize kₜₕ instant value of output voltage and current in each cycle, respectively. k ranges from 1 to N. Pᵢₙₛ(k) stands for the kₜₕ instant powe. [symbol] P̅_{avg}(j) [symbol] _{T}_{ₒ} represents the jₜₕ average output power over one output cycle Tₒ (or over N instantaneous power samples).

In FIG. 38A-B, the distorted output current is approximately divided into 4 linear regions, namely A-D. Compared to regions A and C, the output voltage is relatively small in regions B and D. Accordingly, less arcing is supposed to occur in region B and D, then the lumped load current is small and dominated by the tissue impedance and the interface impedance between tissue and the electrode. Owing to limited voltage and small load current, the yielded instantaneous power is also insignificant. However, more arcing is involved in regions A and C as applied voltage augments, therefore, the lumped load current rapidly surges, as depicted by the sharp current corner in FIG. 38A. The instant powers in these regions have a substantial impact and predominate the average power over a cycle. Provided the dominant impact of regions A and C on overall averaged output power, the load impedance is defined as (9). Due to the similarity between regions A and C, only region A is selected to calculate the load impedance that is exploited later for power adaptation.

|Z|(k)=max(Vₒ(kTₛ))/max(Iₒ(kTₛ)) (9)

where |Z|(k) is the load impedance while max (Vₒ(kTₛ) and max (Iₒ(kTₛ) represent the largest value of sampled output voltage and current within region A, respectively. If needed, then the average value of [symbol] P̅_{avg} [symbol] _{M·T}_{ₒ} and [symbol] |Z| [symbol] _{M·T}_{ₒ} over M cycles can also be defined as (10) and (11).

[symbol]

P̅_{avg} [symbol] _{M·T}_{ₒ}=(Σⱼ₌₁^{M}Σ_{k=(j-1)·N+1}^{(j-1)·N+N}Vₒ(k)·Iₒ(k))/(M·N) (10)

[symbol]

|Z| [symbol] _{M·T}_{ₒ}=(Σₖ₌₁^{M}|Z|(k))/M (11)

where [symbol] P̅_{avg} [symbol] _{M·T}_{ₒ} indicates the averaged output power over M output cycles (or all M N instant power samples) while [symbol] |Z| [symbol] _{M·T}_{ₒ} denotes averaged impedance over M cycles.

Viewing the maximum ADC sampling rate of DSP used for this example, the value of N in (8) is selected as 28 herein. With 28 samples each cycle, the instant output powers are derived as an example for original continuous signals and discretely sampled data as exhibited in FIG. 38A-B. The mean value of power is very close to each other with only 0.3 W arithmetic errors even with the presence of heavy current distortion. This error is conjectured to be much smaller than that using (5).

D. Principle of Impedance-Based Power Adaptation

As stated before, the thermal-feedback-based power adaptive method can reduce collateral tissue damage, but its performance is affected by lots of factors. Therefore, impedance-based ultrafast power adaptation is proposed to conquer the influence of those factors. The principle of this novel methodology is thoroughly explained in this section.

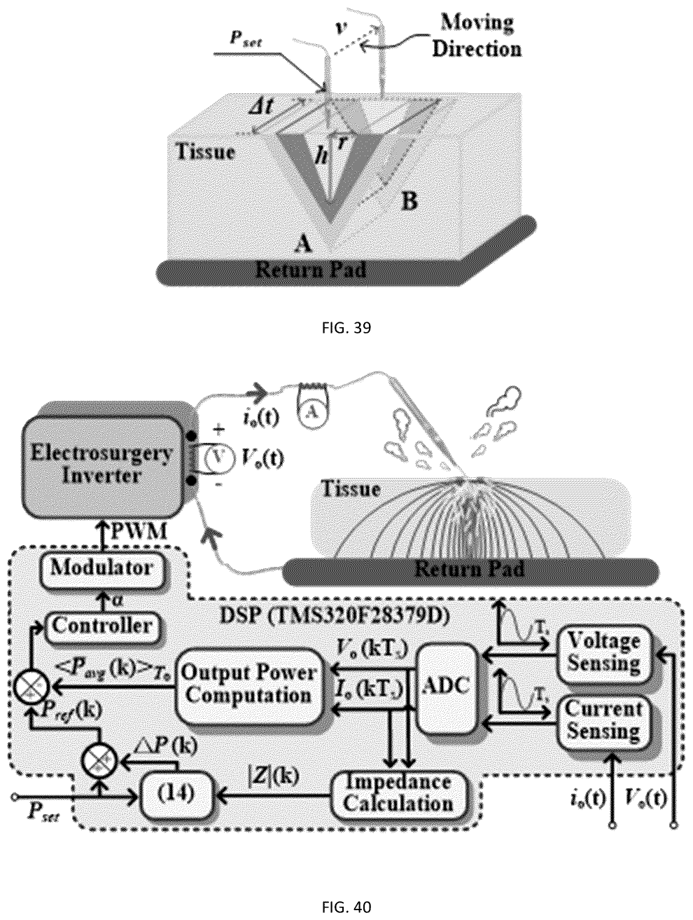

It is reported that as the electrode designed for electrocuting gets in touch with the biotissue, the high-density current flows through the advancing edge of the tissue, followed by gradually decreased current density inside the tissue. The temperature profile of the tissue during electrocuting also quickly drops down as the radial distance measured from the electrode increases. Consequently, only tissue within a few millimeters (mm) radial distance from the electrode is vaporized and removed in the hypothetical shape of an inverted cone. FIG. 37A illustrates the cross-section of such an inverted cone in a silver-gray triangle, and it is redrawn below in FIG. 39 plus many other notations. With the information offered, the ideal energy that induces zero collateral damage when the electrode moves from point A to B is given by (11a):

P_{idl}·Δt=m·c_{eq}·ΔT=½·r·h·v·Δt·ρ·c_{eq}ΔT (11a)

where P_{idl}(W) is the ideal average output power over one cycle, Δt (s) is the cutting time duration and m (kg) is the mass of the targeted tissue. ΔT (K) is the temperature rise while c_{eq} (J·kg⁻¹·K⁻¹) and ρ (kg/m³) are the equivalent specific heat capacity and density of the tissue, respectively. They can be approximately processed as a constant for tissues of similar constituents without causing too many errors.

To simplify analyses, 3 conditions are assumed as follows:
1) The cutting radial distance r is generally quite small and can be assumed as a fixed value.
2) The temperature discrepancies ΔT from normal tissue status to the vaporizing point are fixed, such as from normal body temperature of 37° C. to 100° C.
3) The load impedance magnitude during electrocuting is inversely proportional to the product of electrode insertion depth and moving speed, as depicted by (12a). The (12b) and (12c) are derived when one of the variables in (12a) is fixed.

$$\vert Z \vert = λ \cdot \frac{1}{h \cdot v} \qquad ( 12 a )$$

$$\vert Z \vert = α \cdot \frac{1}{h} \qquad ( 12 b )$$

$$\vert Z \vert = β \cdot \frac{1}{v} \qquad ( 12 c )$$

where λ is the coefficient that relates the |Z| with the h·v (m²/s). α and β is the coefficient bridging the load impedance magnitude |Z| with electrode insertion depth h (m) and moving speed v (m/s), respectively. Instead of function type, different tissue types differ from each other only by separate coefficient values in (12). Condition 3) is experimentally certified for muscle tissue and documented in Section III-E.

Applying (12a) in (11), (13) is elicited as follows:

P_{idl}·|Z|=γ (13)

where γ is a constant and equates to ½·r·λ·ρ·c_{eq}·ΔT.

From (13), the multiplication of ideal power and load impedance magnitude is a constant. To ensure P_{ref}(t) is equal to P_{idl} for reduced damage as load impedance varies, the real-time power adaptation can be generated as (14) and added to preset power reference Pₛₑₜ from surgeons, yielding (15):

$$Δ P ( t ) = γ \cdot ( \frac{\vert {Z}_{0} \vert - \vert Z \vert}{\vert Z \vert \cdot \vert {Z}_{0} \vert} ) \qquad ( 14 )$$

$${P}_{ref} ( t ) = {P}_{set} + Δ P ( t ) \qquad ( 15 )$$

where |Z₀| is equal to γ/Pₛₑₜ. Based on (14) and (15), the impedance-based power adaptation strategy with reduced collateral tissue damage from FIG. 37A is delineated in FIG. 40 together with controller output α and modulator outputs.

### III. Results

A. Sparse-Sampling-Based Power Calculation

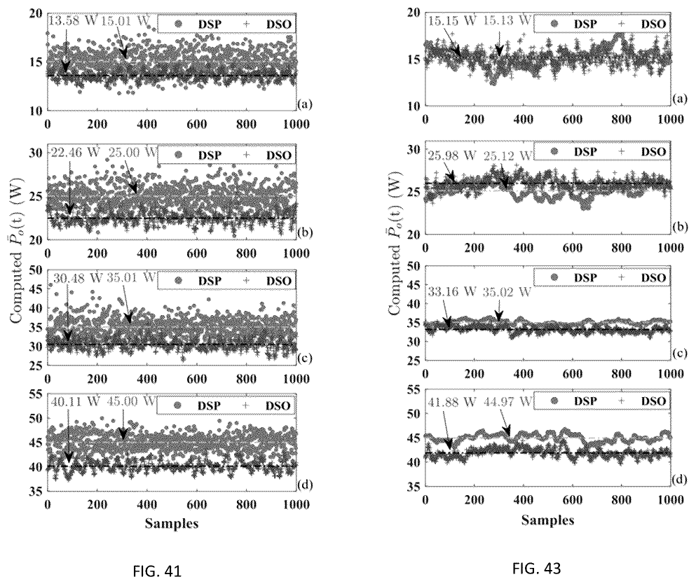

The accuracy of power yielded from the sparse-sampling-based algorithm is experimentally examined by cutting fresh pork using the HFI introduced in Example 2 above. The output power of HFI is under closed-loop control and the power reference in FIG. 41 in graphs (a)-(d) ranges from 15 W to 45 W with a step of 10 W. The averaged output powers obtained using (6) are partially extracted from DSP for 1000 cycles and displayed in FIG. 41 together with those from the digital storage oscilloscope (“DSO”) for different power settings. The data from DSO is sampled at 200 MS/s with sufficient long recording length and then average power is mathematically computed on the basis of those massive data. Therefore, the results from DSO are treated as the most precise reference herein. As concluded from graphs (a)-(d) in FIG. 41, the mean value over all 1000 cycles for DSP and DSO are very close to each other, however, the deviation escalates as the delivered power strengthens. On top of that, instant power in DSP deviates from the average value with relatively large errors for part of the cycles. In another word, the sparse-sampling-based PCA features fairly accurate power computation for most of the cycles, and only a small proportion of samples need improvement.

B. Impact of Arcing Presence on Output Current

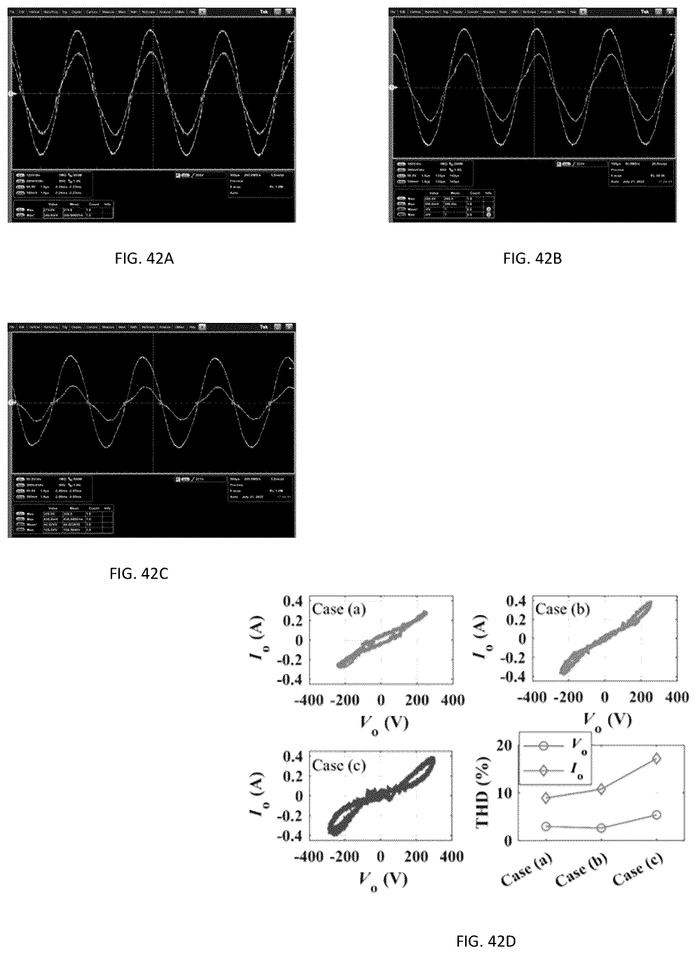

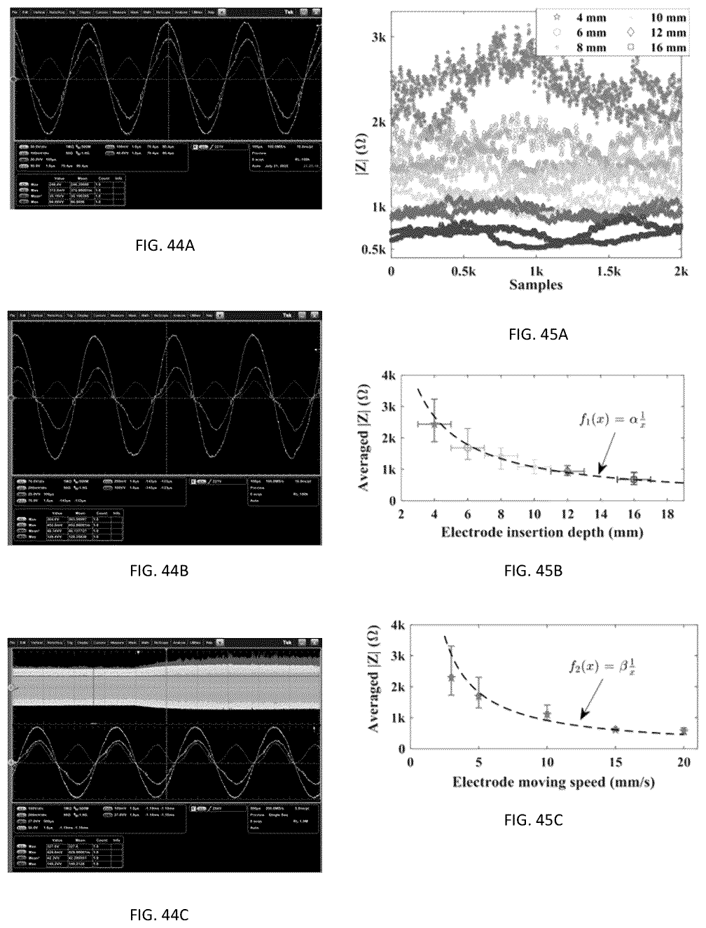

The impact of arcing on output current distortion can be qualitatively probed through revising the output voltage or insertion depth, etc. FIGS. 42A-D experimentally demonstrate different degrees of current distortion during electrocuting. Output current versus voltage is plotted in FIG. 42D for all of them together with the total harmonic distortion (“THD”). As shown in FIG. 42D, for cases (a)-(c) in FIG. 42D, the arcing hysteresis becomes more and more visible as the current distortion escalates. Meanwhile, the current THD is also rapidly aggrandized as manifested by the subfigure in the bottom right corner of FIG. 42D. Nevertheless, the output voltage THD consistently remains at a low level due to the specifically designed filter elaborated in Example 2 above.

C. Multi-Sampling-Based Power Calculation

The computing accuracy performance of the new power quantification method is experimentally checked under the same experimental settings for the sparse-sampling-based approach. The acquired average power from the DSP and those obtained from DSO data are revealed in FIG. 43. Compared with results in FIG. 41, the power values produced by the multi-sampling-based method cluster much closer to the actual values from the DSO. Furthermore, the mean value of power over all 1000 samples from the new method is more accurate with fewer deviation errors than that in FIG. 41. The comparison between power computing accuracy in FIG. 41 and FIG. 43 is summarized in Table I and affirms the necessity of employment of the multi-sampling-based power gauging method if lower power tolerance is of preference.

TABLE I
Power Computation Accuracy Comparison.

| | Deviations | | | |
|---|---|---|---|---|
| | Power Reference | | | |
| Method | @15 W | @25 W | @35 W | @45 W |
| Sparse-sampling-based | 1.43 W | 2.54 W | 4.53 W | 4.89 W |
| Multi-sampling-based | 0.02 W | 0.86 W | 1.86 W | 3.09 W |

D. Steady-State Power Tracking Performance

Utilizing the new power computing method, the steady-state power tracking performance of the HFI in Example 2 above is investigated with a proportional-integral controller and showcased in FIGS. 11A-C. In FIG. 11A, DSO manifests a 35.2 W power averaged over all cycles when the power tracking reference is set as 35 W in DSP. FIG. 11B implies a 48.1 W steady-state power in DSO when the system is tracking 50 W. Combining FIGS. 11A and 11B, it can be declared that the system tracks the preset power reference with tiny errors. FIG. 11C captures the system transitional dynamics, where the power reference is initially set as 35 W, and then, directly shifted toward 50 W.

E. Relationships of Load Impedance Against the Electrode Insertion Depth and Cutting Speed

To authenticate condition 3) mentioned in Section II-D, electrocuting trials with various insertion depths and cutting speeds are conducted with the help of a programmable Emile3 3-axes robotic gantry. Experimental results are documented in FIGS. 45A-B. FIG. 45A presents the secured load impedance defined in (9) versus the electrode insertion depth. In the middle subplot, electrode insertion depth varies from 4 mm to 16 mm with a±1 mm error bar, and the midpoint of load impedance over 2000 samples is graphed on the vertical axis. It is observed that the earned load impedance versus electrode insertion depth h approximately fits an inverse proportional function ƒ₁(x). In the bottom subplot, the load impedance is plotted versus the electrode moving speed v, and the curve roughly fits the inverse proportional function ƒ₂(x) if the cutting speed is not too slow. Combining both functions ƒ₁(x) and ƒ₂(x), the load impedance is deduced as an inverse proportional function of h·v that is in the form of (12a). Therefore, the experiment results in FIGS. 12A-C advocate the validity of condition 3) in Section II-D.

F. Impedance-Based Power Adaptation

By following (13), the power reference is so adjusted such that the product of P_{ref}(t) and |Z| is brought back to γ as |Z| varies. In this way, the actual electrocuting is near to ideal cutting with minimal collateral tissue damage. It is noteworthy that the exact value of γ is hard to theoretically calculate, therefore, its approximated optimal value is figured out by extensive experiments. Based upon massive trials, the value of γ used in this paper is 30000, and all load impedance in DSP is smoothed out by the approach of moving average. The process of moving averaging enhances system power tracking stability by eliminating power reference violent fluctuation that originates from load impedance sudden jump. The larger the moving average window length, the better tracking stability. However, it also compromises prompt power adaptation dynamics by slowing it down. To keep a balance, the load impedance in this example is monitored each cycle, but all impedance values used in (14) for the power reference regulation are their moving average scrolling over 10 cycles. Based on that, the efficacy of the load impedance-based power adaptation method is scrutinized with multi-sampling-based output power computation in FIG. 40, and 5 test scenarios are tabulated and listed in Table II.

TABLE II
CONFIGURATIONS FOR 5 TEST SCENARIOS.

| | Trials | | | | | | | | | |
|---|---|---|---|---|---|---|---|---|---|---|
| | Test Scenarios | | | | | | | | | |
| Settings | #1 | | #2 | | #3 | | #4 | | #5 | |
| Pₛₑₜ | 35 | W | 35 | W | 35 | W | 35 | W | 35 | W |
| Cutting Speed | 5 | mm/s | 5 | mm/s | 10 | mm/s | 5 | mm/s | 10 | mm/s |

| Insertion Depth | Shallow | | Shallow | Shallow | Deep | Deep |
|---|---|---|---|---|---|---|
| ΔP(t) | 0 | W | Using (14) | Using (14) | Using (14) | Using (14) |
| P_{ref}(t) | 35 | W | Using (15) | Using (15) | Using (15) | Using (15) |

In test scenario 1, the cutting speed is 5 mm/s and set power is kept the same at 35 W during the entire electrocuting. The electrode is fixed on the programmable Emile3 3-axes robotic gantry and its moving speed is under control for the sake of experimental repeatability. Instead of constant power configuration, test scenarios 2-5 are equipped with power adaptation. And their cutting speeds along with electrode insertion depth are intentionally configured differently to examine the general applicability of the proposed impedance-based power adaptation philosophy. The purpose of test scenario 1 is to emulate conventional electrosurgery and it also provides a reference criterion of collateral tissue damage for test scenarios 2-5 as explained later in Table III.

TABLE III
EVALUATION OF ELECTROCUTING QUALITY.

| | Trials | | | | |
|---|---|---|---|---|---|
| | Test Scenarios | | | | |
| Metrics | #1 | #2 | #3 | #4 | #5 |
| Spread at L1 | 2.18 | 0.75 | 0.48 | 1.00 | 0.32 |
| Spread at L2 | 1.63 | 1.27 | 0.35 | 1.10 | 0.34 |
| Spread at L3 | 0.97 | 0.48 | 0.55 | 1.25 | 0.42 |
| Spread at L4 | 1.20 | 0.96 | 0.37 | 1.12 | 0.39 |
| Averaged | 1.49 | 0.86 | 0.43 | 1.11 | 0.36 |
| Spread | | | | | |

Note:
All measurements are gauged in millimeters (mm).

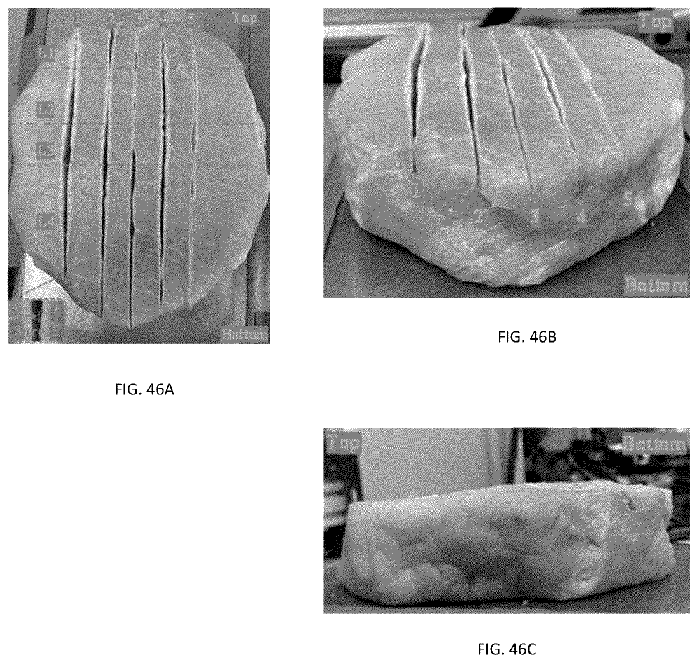

Following the experimental setting listed in Table II, fresh pork is cut from the top to bottom, and 5 electrocuting traces are outcomes, as present in FIGS. 46A-C. From FIG. 46C, it is seen that the biotissue surface is not flat, and it is often the case in actual electrosurgery. Thus, it is reasonable to vary the electro-de insertion depth with a noticeable step to investigate the performance of the proposed impedance-based power adaptive approach. The quality of cutting trace is evaluated in terms of thermal spread which is exemplified in FIG. 46A. The thermal spreads of all 5 cutting traces are measured at 4 cross points with lines 1-4, namely, L1-L4. The narrower the thermal spread, the better the cutting quality, since less thermal spread signifies reduced collateral tissue damage.

The gauged thermal spread is summarized in Table III. As noticed in Table III, test scenario 1 conducted with constant power has maximum thermal spread. With the impedance-based power adaptation, the thermal spread of test scenario 2 is significantly reduced from test scenario 1. When the electrode insertion depth exceeds more than half of the entire electrode blade, as in scenario 4, the power adaptation can still reduce thermal spread, but the reduction is not as much as that in a shallow case much like test scenario 2. But in actual electrosurgery, the electrode insertion depth is controlled by surgeons with a routinely shallow insertion depth (around 6 mm) since deep cutting can be achieved by several shallow slices. Looking through test scenarios 2 and 3 or test scenarios 4 and 5, it is verified that the proposed impedance-based power adaptation works well even if cutting speed varies. For test scenarios 2 and 4 (or test scenarios 3 and 5), the cutting speed is maintained at 5 mm/s (or 10 mm/s) while the electrode insertion depth differs. It is seen that the thermal spread is always smaller for traces cut at 10 mm/s than that at 5 mm/s. It underlines the important role of cutting speed in reducing thermal spread once more. Aside from that, it might also forecast the necessity to massively collect habitual cutting speeds preferred by surgeons such that the value of γ is accordingly optimized for reductive thermal spread.

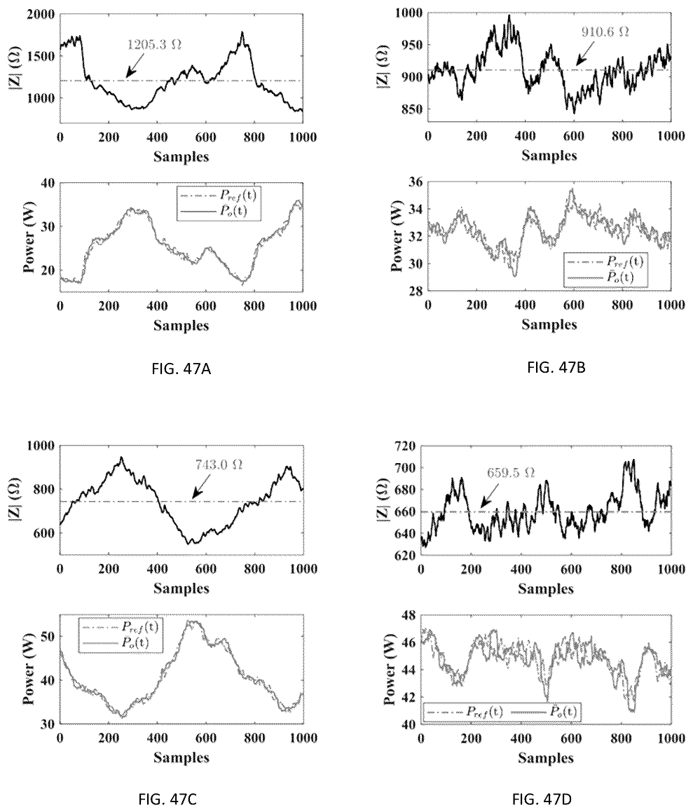

As seen in (14) and (15), the proposed impedance-based power adaptation method supervises the load impedance in real time and adjusts the power reference cycle by cycle. It implies that power reference updates in a few microseconds in consideration of 390 kHz output frequency. It is much faster than the thermal-based power adaptive manner. Fragment of power references during electrocuting together with measured load impedance for test scenarios 2-5 are plotted in FIG. 47A-D, respectively. In FIGS. 47A-D, the power reference P_{ref}(t) is finely tuned per load impedance and it is also closely followed by system average output power P̅ₒ(t). Thereby, it proves the feasibility of the impedance-based power adaptation technique and also validates the ultrafast power tracking capability of the entire high-frequency inverter system.

### IV. Discussion

This example briefly illustrates the current paths inside the biotissue and elucidates the electrocuting mechanism to pave the way for a basic understanding of electrosurgery. Conventional electrosurgery delivers constant power to the target tissue with high accuracy. It increases the possibility of added collateral tissue damage. Surgeons can frequently and manually modify power settings to better cutting quality with less tissue damage. At the cost of doing that, the termination of time-sensitive electrosurgical processes is unavoidable and lengthens the clinical duration which might lead to serious consequences or cause potential exposure to a higher risk of clinical surgical failure. Therefore, autonomous real-time power adaptation is of paramount importance.

With the information on tissue surface temperature, the thermal-feedback-based power adaptation can reduce tissue collateral damage during electrosurgery. But it requires one extra thermal sensor and resultant cost, heavy thermal data processing, fast communication, etc. Besides that, it is also experimentally revealed in this example that the accuracy of temperature measurement is notably affected by thermal sensor mounting locations. Moreover, thermal sensor resolution and the existence of smoke during electrosurgery impose an impact on the thermal measurement precision as well. Because of that, either a costly thermal sensor with good resolution or a smoke evacuation pencil is needed to get rid of those impacts. Additionally, an advanced thermal sensing compensating algorithm might be another alternative to tackle the smoke issue for thermal sensing. Nevertheless, with the constraint of thermal sensor refresh rate, the thermal-based power adaptation can hardly refresh the power reference beyond 100 Hz, which sets a barrier to thermal sensor application in circumstances requiring ultrafast power adjustments. To crack all limitations or downsides related to the thermal-based power adaptive approach, the load impedance-based power adaptation method is brought forward in this example. Before the detailed explanation of such method, two schemes to calculate average output power with limited output measurements are formulated.

The sparse-sampling-based method requires the assumption of sinusoidal output voltage and current, which is not always true for actual electrosurgery due to the presence of arcing. But it dramatically shrinks the sampling requirements and calculates output power with only one voltage sample and two current samples each cycle. The power calculation results are analyzed through comparison with those from the digital storage oscilloscope which are thought of as the most accurate. It turns out that the sparse-sampling-based method has satisfied power computing accuracy and only a small proportion of points are of relatively large errors. Therefore, the sparse-sampling-based tactic is suited for the case with limited processor computing power or with low accuracy concerns. It might need improvement for the application that is sensitive to power precision. This example experimentally evidences the presence of arcing during electrocuting and demonstrates the current distortion. The distortion largely depends on the amount of arcing and the output current is no longer sinusoidal when heavily distorted. This observation accounts for relatively large power calculation errors seen in some of the power points quantified by the spare-sampling-based method.

Instead of sparse samples, the multi-sampling-based approach divides one output cycle into 4 linear regions for load impedance definition and calculates the output power with 28 samples per cycle. Experimental results show that the multi-sampling-based power computation values cluster very close to the values from the digital storage oscilloscope and have better power counting fidelity than that of the sparse-sampling-based formula. However, Table I reveals that power calculation errors still remain for the multi-sampling-based method with 28 samples of both outputs.

As is well-known, the greater the sample number N per cycle, the more accurate the power calculation. Therefore, a sampling number N larger than 28 is needed to further reduce power computing errors. But larger N requires a higher sampling rate that is limited by the maximum achievable sampling speed on the ADC. Moreover, the computation burden also rapidly escalates as the sampling rate increases. The output frequency herein is 390 kHz with a rough period of 2.5 μs, therefore, the digital computation speed is another key factor. In consequence, a tradeoff among power calculating accuracy, digital computing burden, and speed restriction should be maintained for satisfactory performance.

Furthermore, the cycle number M in (10) poses an impact on power tracking performance in which a smaller M features prompt power tracking, but it also compromises system control stability. On the other side, an overlarge M ensures system stability whereas it also jeopardizes system tracking dynamics. And thus, the value of M should be properly selected to achieve a balance between prompt power response and tracking stability. In viewing of typical availability of maximum ADC sampling rate and ADC channels from low-end industrial-scale DSPs, the output current is sampled 14 times each cycle by two ADC channels in this paper. The sampling initiation point of each channel is so arranged such that all 28 sampling points are evenly distributed in time without overlapping. The less distorted output voltage is sampled 14 times each cycle and another 14 sampling points are interpolated between two actual samples. In other words, N in (8) equates to 28 in this example.

If more sampling points are crucial for ultrahigh power computation precision, then a dedicated data acquisition system or high-end FPGA with a very high ADC sampling rate is an alternative solution. With the rapid advancement of technology, normal digital processors might become sufficiently powerful to handle this task in the near future. Nevertheless, the multi-sampling-based approach is more suitable for circumstances that have sufficient digital computing capability and need high fidelity as well.

Given the characteristics of the two methods, power counting is dependent on the actual circumstances presented in a given operating environment. In this example, the way of multi-sampling is embraced by viewing the importance of power fidelity in electrosurgery.

Based on the definition of impedance, the relationships of load impedance against electrode insertion depth and cutting speed are established, followed by experimental proofs. Both correlations fit in the form of inverse proportional function. But it should be noted that load impedance against the electrode cutting speed seems to saturate and diverge from the inverse proportional form when the speed is too small. To that end, data collection may prove useful to gather individual surgeons' preferred cutting speed together with electrode insertion depth. Doing so would provide more accurate links among impedance, insertion depth, and cutting speed to refine the power control method.

In this example, muscle tissue is used and its parametric properties, such as mass density, etc. are assumed to be uniform and there is no local variation. Even in this framework, it is still quite challenging to theoretically quantify the precise value of γ in (13) since the biotissue specific heat capacity is reported as a function temperature in the literature. On that account, it is also challenging to determine the value of equivalent specific heat capacity c_{eq}. Before c_{eq} can be precisely determined, there is a need of extensive clinical trials to experimentally determine the approximated optimal value of γ for different tissue types. On the foundation of relationships just built, the impedance-based power adaptation is developed. This novel approach takes the thermal sensor away and updates the power reference in a cycle-by-cycle ultrafast manner, namely, in less than 3 μs in this example. It is orders of magnitude faster than the thermal-feedback-based power adaptation. Therefore, the proposed method is extremely suitable for cases that are very sensitive to power mismatch and need ultrafast power modulations, such as electrosurgery on the brain, cerebral vessels, heart, etc. If rapid power adaptation is not required, then it is also easy to slow down the power reference update rate by reducing load impedance monitoring frequency.

### V. Conclusion

This example details and evaluates two novel ways of computing output power using limited output measurements for electrosurgery. The sparse-sampling-based method only samples output voltage once and current twice whereas it yields output power computation with small deviation errors. With slightly increased sampling points and computation burden, the multi-sampling-based method improves accuracy even when nonlinearity from electrosurgical arcing is present on the outputs. These two methods are implemented in low-end industrial-scale processors with limited sampling speed. Consequently, they may reduce the need for high-end processors with fast sampling speed and save corresponding costs for applications involving high-frequency and highly distorted outputs.

In addition, a relationship among defined load impedance, electrode insertion depth, and cutting speed is developed from the multi-sampling-based methodology. Evolving from the relationship, an original impedance-based power adaptation strategy is further formulated with the capability of autonomous power reference updating in a cycle-by-cycle ultrafast manner. Experimental results of different test scenarios show that electrocuting traces delivered by the impedance-based power adaptation strategy yield diminished thermal spreads from conventional electrosurgery, which validates the feasibility and efficacy of the impedance-based power adaptation strategy.

Moreover, this power adaptation strategy eliminates the thermal sensor and eradicates the drawbacks associated with thermal-based power adaptation. In other words, collateral tissue damage in terms of thermal spread is reduced with the extra benefits of less sensor count and decreased cost. This demonstrates the superiority of impedance-based power adaptation over existing thermal-based power adaptation strategy.

The description of different advantageous arrangements has been presented for purposes of illustration and description and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to best explain the principles of the examples, the practical application, and to enable others of ordinary skill in the art to understand the disclosure for various examples with various modifications as are suited to the particular use contemplated.

1. An electrosurgical system, comprising:
a high-frequency inverter (“HFI”) having a full bridge;
a control system electrically coupled to the HFI that controls output parameters including one or more of an output power Pₒᵤₜ(t) and an output voltage or current by varying power reference P_{ref}(t) or switch states of the HFI, wherein the control system causes a power adaptation ΔP(t) to a preset power Pₛₑₜ based on receiving at least one of impedance feedback and thermal feedback according to the following relationship:
P_{ref}(t)=Pₛₑₜ+ΔP(t).

2. The electrosurgical system according to claim 1, further comprising a multi-resonant-frequency (“MRF”) filter electrically coupled to the HFI;
wherein the MRF filter comprises a first resonant tank and a second resonant tank, wherein the first resonant tank resonates at a switching frequency and the second resonant tank resonates at least at third-, fifth-, and seventh-order harmonics; and
wherein a fundamental output frequency of the HFI is the same as a switching frequency of the HFI.

3. The electrosurgical system according to claim 2, wherein the switching frequency is 390 kHz.

4. The electrosurgical system according to claim 2, wherein the HFI generates a bipolar square waveform, and wherein the MRF filter shapes the bipolar square waveform into a sinusoidal waveform output, wherein a transformer primary side voltage of the HFI is determined based on the following:
$${V}_{p} ( t ) = \frac{4 {V}_{i n}}{π} \cdot cos ( α ) \cdot sin ( 2 π {f}_{s} t ) .$$

5. The electrosurgical system according to claim 1, further comprising:
an electric scalpel electrically coupled to a transformer secondary side of the HFI; and
a return pad electrically coupled to the transformer secondary side of the HFI, wherein the return pad is configured to receive a load in the form of biomedical tissue that permits current flow therethrough from the electric scalpel to the return pad thereby closing a path for the current flow.

6. The electrosurgical system according to claim 1, further comprising:
a thermal sensor electrically coupled to the control system, wherein the thermal sensor is configured to detect a surface temperature of a load.

7. The electrosurgical system according to claim 1, wherein the control system comprises a modulator configured to output pulse-width modulation signals to the HFI, and a power controller that tracks the output power reference P_{ref}(t).

8. A method for using the electrosurgical system of claim 1, the method comprising:
receiving, via the control system, at least one signal with an indication of thermal feedback and/or impedance feedback;
determining, via the control system, a power adaptation ΔP(t) based on the thermal feedback and/or impedance feedback; and
combining, via the control system, a preset power Pₛₑₜ for the HFI with the determined power adaptation ΔP(t) to obtain the output power reference P_{ref}(t) for the HFI.

9. The method of claim 8, wherein receiving, via the control system, the at least one signal with the indication of the thermal feedback and/or the impedance feedback comprises:
receiving, via the control system and per each switching cycle, at least one signal indicating values for a plurality of pairs of output voltage and output current that are measured simultaneously during a given switching cycle.

10. The method of claim 8, further comprising:
monitoring, via the control system, the output power Pₒᵤₜ(t) and thereby tracking the output power reference P_{ref}(t).

11. The method of claim 9, further comprising:
determining, via the control system, an ideal average output power P_{idl} based on a cutting time duration Δt, a mass m of the load, a temperature rise ΔT of the load, a specific heat capacity c_{eq} of the load, a density ρ of the load, an electrode insertion depth h, and/or a cutting width r, as set forth below:
P_{idl}·Δt=m·c_{eq}·ΔT=½·r·h·v·Δt·ρc_{eq}·ΔT.

12. The method of claim 11, wherein determining, via the control system, the power adaptation ΔP(t) based on the thermal feedback and/or impedance feedback comprises:
determining, via the control system, a load impedance based on a largest value of sampled output voltage and output current for the given switching cycle; and
determining, via the control system, the power adaptation ΔP(t) based on the load impedance and the ideal average output power P_{idl}.

13. The method of claim 12, wherein determining the power adaptation ΔP(t) is further based on a load impedance value determined from a moving average of the determined load impedance values over at least 10 switching cycles.

14. The method of claim 8, further comprising:
updating, via the control system, the output power reference P_{ref}(t) for the HFI for each switching cycle in 3 μs or less.

15. The method of claim 8, wherein receiving, via the control system, the at least one signal with the indication of the thermal feedback and/or the impedance feedback comprises:
receiving, via the control system and per each switching cycle, at least one signal indicating an output voltage Vₒ(t) corresponding to an output voltage positive peak at Tₛ/4 and a first and a second sample of output current, wherein the first sample of output current iₒ(k) is measured between 0 and Tₛ/4 and the second sample of output current iₒ(k+1) is measured after the first sample output current such that the first and the second output current samples do not overlap in time.

16. The method of claim 8, further comprising:
generating, via the HFI, a bipolar square waveform; and
shaping the bipolar square waveform into a sinusoidal waveform output, via a MRF filter electrically coupled to the HFI, wherein the MRF filter comprises a first resonant tank and a second resonant tank, wherein the first resonant tank resonates at a switching frequency and the second resonant tank resonates at least at third-, fifth-, and seventh-order harmonics, and wherein a fundamental output frequency of the HFI is the same as a switching frequency of the HFI.

17. The method of claim 16, further comprising:
determining, via the control system, a transformer primary side voltage of the HFI electrically coupled to the MRF filter based on the following:
$${V}_{p} ( t ) = \frac{4 {V}_{in}}{π} \cdot cos ( α ) \cdot sin ( 2 π {f}_{s} t ) .$$

18. The method of claim 8, further comprising:
adjusting, via the control system, a phase shift angle α₀ between gate signals of diagonal switch pairs of the HFI based on the following relationship:
$${α}_{0} = f ( {V}_{ref} ) = \frac{180}{π} \cdot {cos}^{- 1} ( \frac{π \cdot {V}_{ref}}{4 \cdot n \cdot {V}_{in}} ) .$$

19. The method of claim 8, further comprising
continuously monitoring, via the control system, a surface temperature of a load;
determining, via the control system, that the surface temperature of the load differs from a predetermined nominal tissue temperature; and
adjusting, via the control system, the power reference P_{ref}(t) based on the relationships:
$${P}_{ref} ( t ) = {P}_{set} + Δ P ( t )$$
$$ΔP ( t ) = {P}_{set} \cdot ( \frac{{T}_{nom}}{max ( {T}_{tissue} ( t ) )} - 1 )$$
such that the surface temperature of the load is controlled towards the predetermined nominal tissue temperature.

20. A non-transitory computer-readable medium having stored thereon program instructions that upon execution by a processor, cause performance of a set of steps comprising:
the control system receiving at least one signal with an indication of thermal feedback and/or impedance feedback;
the control system determining a power adaptation ΔP(t) based on the thermal feedback and/or impedance feedback; and
the control system combining a preset power Pₛₑₜ for the HFI with the determined power adaptation ΔP(t) to obtain the output power reference P_{ref}(t) for the HFI.
